SEQUENCE LISTING (7 sequences)

SEQ ID NO: 1 - protein, 30 aa, Artificial Sequence
  Inventor Tanihara, Masao Inventor Otsuki, Chikara Inventor Mikami, Hiroshi Inventor Kinoshita, Hisao
  Description of Artificial Sequencepeptide
PPGPPGPPGPPGPPGPPGPPGPPGPPGPPG

SEQ ID NO: 2 - protein, 15 aa, Artificial Sequence
  Description of Artificial Sequencepeptide
PPGPPGPPGPPGPPG

SEQ ID NO: 3 - protein, 30 aa, Artificial Sequence
  Description of Artificial Sequencepeptide
  MISC_FEATURE (1)..(30): Xaa is defined as 4Hyp
PXGPXGPXGPXGPXGPXGPXGPXGPXGPXG

SEQ ID NO: 4 - protein, 10 aa, Artificial Sequence
  Description of Artificial Sequencepeptide
VPGVGVPGVG

SEQ ID NO: 5 - protein, 15 aa, Artificial Sequence
  Description of Artificial Sequencepeptide
  MISC_FEATURE (1)..(15): Xaa is defined as 4Hyp
PXGPXGPXGPXGPXG

SEQ ID NO: 6 - protein, 30 aa, Artificial Sequence
  Description of Artificial Sequencepeptide
  MISC_FEATURE (1)..(30): Xaa is defined as 4Hyp
PXGPXGPXGPXGPQGIAGPXGPXGPXGPXG

SEQ ID NO: 7 - protein, 30 aa, Artificial Sequence
  Description of Artificial Sequencepeptide
  MISC_FEATURE (1)..(30): Xaa is defined as 4Hyp
PXGPXGPXGPXGPLGIAGPXGPXGPXGPXG

(19) United States
(12) Patent Application Publication
(10) Pub. No.: US 2007/0207180 A1
(43) Pub. Date: Sep. 6, 2007

(54) Synthetic polypeptide-containing bioapplicable material and film-forming material

(76) Inventors: Masao Tanihara, Kyoto-shi (JP); Chikara Otsuki, Ikoma-shi (JP); Hiroshi Mikami, Kasugai-shi (JP); Hisao Kinoshita, Ikoma-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/365,832

(22) Filed: Mar. 2, 2006

(51) Int. Cl. A61K 38/16; C07K 7/08; C07K 7/06

(52) U.S. Cl. 424/423; 530/324; 530/326; 530/327; 530/328; 530/329; 530/330; 530/331

(57) ABSTRACT

The present invention provides a bioapplicable material (or composition) or film-forming material (or composition), which is free from a risk of an infection by a pathogenic organism or a transmission of a causative factor, has a high safety. The material (or composition) comprises a collagen-like synthetic polypeptide having at least an amino acid sequence represented by the formula -Pro-Y-Gly- (wherein Y represents Pro or Hyp). The polypeptide may show positive Cotton effect at a wavelength in range of 220 to 230 nm and negative Cotton effect at a wavelength in range of 195 to 205 nm in a circular dichroism spectrum. At least part of the polypeptide may be capable of forming a triple helical structure. The polypeptide may be degradable with a collagenase.

### FIELD OF THE INVENTION

The present invention relates to a bioapplicable composition which is free from a risk of an infection by a pathogenic organism or that of a contamination by a causative (or pathogenic) factor, has a high safety, contains a synthetic polypeptide capable of forming a structure similar to a collagen, and is excellent in biocompatibility [e.g., a biomaterial (or a biocompatible material) useful for various applications such as a wound-coating material; a cosmetic preparation; a food composition useful for an animal feeding stuff or a food; and a pharmaceutical preparation composition], and a film-forming composition containing the synthetic polypeptide [e.g., a coating material (or a covering material) such as a paint, a coating agent (or a coating flux), a paste, a finishing (or surface-treating) agent, or a processing agent, and an adhesive (or an adhesive agent)].

### BACKGROUND OF THE INVENTION

Biomaterials (particularly medical biomaterials) are required to have bioaffinity, and compatibility to body fluid (e.g., blood) or tissue, and to have no toxicity or antigenicity. From such view points, a collagen, which is derived from a living body, is excellent in bioaffinity and biocompatibility and is degradable and sorbable (or absorbable) in a living body, has been widely utilized.

A collagen is a fibrous protein found in all multicellular animals. The collagen is a main component of skins or bones, and occupies 25% of total proteins in mammals. A typical collagen molecule has a rope-like superhelical structure, which is referred to as a triple helical structure, comprising three collagen polypeptide chains. The polypeptide chains having the above-mentioned triple helical structure can self-assemble to form a fibril having a length of several nanometers to several tens nanometers. Further, these fibrils can be arranged to form a fiber structure having a length of several nanometers to several tens nanometers.

As methods for using a collagen as a biomaterial, there may be mentioned, for example, a method of grafting or transplanting an intact or lyophilized skin tissue derived from a pig on a skin area damaged by a burn or scald, a method of removing cellular components from a tissue with enzyme treatment, and a method of using a collagen which is solubilized by a treatment with an acidic solution or an enzyme to reconstitute a desirable form.

There are various suggestions for utilizing a collagen as a biomaterial. For example, Japanese Patent Application Laid-Open No. 292716/1994 (JP-6-292716A) discloses a medical material in which a coating layer of a collagen (a porous or sponge-like collagen) is formed on a surface of a biodegradable and biosorbable (or bioabsorbable) material (e.g., a polyglycolic acid, a glycolic acid-lactic acid copolyester, and a mixture of a polyglycolic acid and a polylactic acid). This document mentions that an alkali-soluble collagen or an enzyme-soluble collagen is preferred to reduce antigenicity.

Japanese Patent Application Laid-Open No. 180820/2003 (JP-2003-180820A) discloses a biomaterial for implanting in a living body, which comprises a base, and a tissue-inducing material (e.g., a collagen ingredient, or a collagen) binding to a surface of the base material and having a tissue-inducing ability.

Japanese Patent Application Laid-Open No. 271207/2000 (JP-2000-271207A) discloses an antiadhesive membrane (a sponge-like or film-like membrane) which can be sutured, wherein a coating layer containing a gelatin or a hyaluronic acid is formed on a surface of a nonwoven fabric layer made of a collagen fiber. This document also describes that the coating layer may contain a crosslinked gelatin or hyaluronic acid, and the collagen, gelatin and hyaluronic acid is preferably derived from a living body.

Japanese Patent Application Laid-Open No. 291937/1998 (JP-10-291937A) discloses a wound-coating material which contains a specific peptide and at least one member selected from the group consisting an alginate gel, a collagen, a fibrin, a chitin, a chitosan, and a derivative thereof. This document mentions that a cowskin-derived or pig skin-derived collagen is suitable as the collagen.

Japanese Patent Application Laid-Open No. 327520/2001 (JP-2001-327520A) discloses a medical adhesive tape in which an a thermosensitive biodegradable and biosorbable adhesive layer which expresses or increases adhesion property at a temperature of not lower than a body temperature, or a water-sensitive biodegradable and biosorbable adhesive layer which expresses or increases adhesion property through the contact with water is formed on at least one surface of a tape substrate made of a biodegradable and biosorbable polymer. This document also mentions a microfibril collagen may be contained in such an adhesive layer. Moreover, Japanese Patent Application Laid-Open No. 290633/2000 (JP-2000-290633A) discloses an adhesive agent for biological tissue, which comprises a paste containing a partial hydrolysis product of a collagen protein (e.g., a gelatin) and a water-soluble chitin derivative as main components, and a crosslinking agent containing a bi- to polyfunctional aldehyde (e.g., glyoxal, succinaldehyde, glutaraldehyde, and malealdehyde) as a main component. This document mentions that the origin of the gelatin is clear and the gelatin is free from contamination with a pathogenic organism of bovine spongiform encephalopathy.

Such a collagen to be used in these medical materials is derived from a mammal, and usually, a collagen derived from bovine or pig is employed as a raw material of a biomaterial in many cases. On the other hand, a causative substance of sheep tremor or bovine spongiform encephalopathy is an infectious protein called as prion, and the infectious protein is considered as one of causes of human Creutzfeldt-Jakob disease infection. The prion is a protein, and it is indicated that the prion is difficult to deactivate with a conventional pasteurization or sterilization method, further that prion is infectious over species (Nature Review, Vol. 2, pp. 118 to 126, 2001).

Accordingly, there have been always existed the risk of an infection (or a transmission) to pathogenic organisms or a causative factor such as prion which cannot be removed by conventional pasteurizations or sterilizations. As described in JP-2000-290633A, it is necessary to confirm the existence of pathogenicity. Moreover, in order to avoid a risk of an infection of a pathogenic organism, Japanese Patent Application Laid-Open No. 041425/1996 (JP-08-041425A) discloses a method for removing a prion in a collagen derived from an animal or human being, which comprises removing a cell and tissue piece from a collagen solution, and subjecting the solution with an alkali; and a collagen obtained by the method. However, such a process requires confirmation of the safety and is complicated, resulting in increase of costs.

Moreover, since various cell adhesion sites are found in a naturally occurring collagen, the naturally occurring collagen cannot exert cell selectivity for any applications. For example, in the case using a collagen as a material for inducing a nerval axon, migration or growth rate of surrounding fibroblast is faster than elongation rate of the axon, resulting in forming scarring tissue, and the axon cannot be elongated. It is therefore necessary to take a step to cover around the collagen with a material for protecting migration of fibroblast, or others.

Further, regarding methods for chemical synthesis of collagen analogues, it has been reported that a soluble polyamide having a molecular weight of 16,000 to 21,000 is obtained by dissolving a p-nitrophenyl ester of a peptide represented by the formula: Pro-Ser-Gly, or a p-nitrophenyl ester of a peptide represented by the formula: Pro-Ala-Gly in dimethylformamide, adding triethylamine thereto, and allowing to stand the mixture for 24 hours (J. Mol. Biol., Vol. 63, pp. 85 to 99, 1972). In this literature, the soluble polyamide is estimated to form a triple helical structure based on the circular dichroism spectra. However, there are not referred to properties of the obtained polymer.

It also has been reported that a method for obtaining a polyamide, which comprises dissolving a 50-mer peptide containing the sequence Val-Pro-Gly-Val-Gly derived from elastin in dimethyl sulfoxide, adding 2 equivalents of 1-ethyl-3-(3-dimethylaminopropyl)-carbodiimide, 1 equivalent of 1-hydroxybenzotriazole and 1.6 equivalents of N-methylmorpholine thereto, allowing to stand the mixture for 14 days, and dialyzing the resultant mixture with a dialysis membrane (molecular weight cut-off: 50,000) (Int. J. Peptide Protein Res., Vol. 46, pp. 453 to 463, 1995).

On the other hand, Japanese Patent Application Laid-Open No. 142124/1994 (JP-6-142124A) discloses an biocompatible implant member which comprises an aliphatic polyester obtained by use of a microorganism or a chemical synthesis.

Such an aliphatic polyester, however, is inferior in bioaffinity or biocompatibility to a collagen derived from animals.

Moreover, a collagen has a high moisturizing effect, and is widely used in many applications for cosmetic preparation, including a basic cosmetic preparation and a makeup cosmetic preparation. In general, a collagen derived from bovine or pig is used as a raw material of a cosmetic preparation in practical cases. Among them, a collagen derived from bovine has been in heavy usage because of excellent physicochemical properties and less smell.

However, as described above, a risk of a mammal-derived collagen has been pointed out. As alternatives to a collagen derived from bovine or others, a collagen derived from fish or the like has been proposed. For example, Japanese Patent Application Laid-Open No. 302690/2001 (JP-2001-302690A) discloses a tripeptide which has a high skin-penetrative property and is useful for an external preparation such as a cosmetic preparation, and the tripeptide has a specific amino acid sequence obtained by specifically decomposing a collagen component or gelatin component derived from bone, skin or tendon (or sinew) of bovine or pig, or fish skin with a collagenase enzyme.

The collagen derived from fish, however, sometimes gives out a stench depending on formulation of a cosmetic preparation, or others. Japanese Patent Application Laid-Open No. 146835/2003 (JP-2003-146835A) discloses a cosmetic preparation containing a fish-derived collagen, a lipophilized powder, and a silicone, wherein the collagen is coated with a silicone coat to inhibit generation of stench. However, in such a method, the moisturizing action of the collagen cannot be effectively utilized because of the silicone coat formed around the collagen. Further, since such a preparation essentially requires the powder, the preparation can be used only in the limited application.

Moreover, a collagen or a treated product thereof (e.g., a decomposed product of a collagen, and a hydrolysate of a gelatin obtained by heat-denaturing a collagen) has been widely utilized as a raw material for a food. In particular, it has been known that oral intake of a collagen brings about various effects, e.g., a pharmacologic effect such as an action for growth promoting osteoblast, an action for reinforcing bone, an action for promoting metabolism of biological tissue against aging, an action for promoting cutaneous metabolism, or an action for activating skin.

On the other hand, as a raw material of a collagen, a material derived from a mammal such as bovine or pig has been used. For example, JP-2003-180820A discloses a food containing a concentrate of a purified collagen obtained by purifying an extracellular matrix derived from human being or an animal. In the case of taking such a food, a large quantity of a highly absorbable collagen can be taken, and an extracellular matrix generation in a living tissue is stimulated and facilitated. The generated extracellular matrix acts on an undifferentiated cell in a living tissue, facilitates proliferation or differentiation of the cell, and activates the living tissue, and resulting in festinating regeneration of an affected area. JP-2001-302690A discloses a highly absorbable food containing a tripeptide in a proportion of not less than 0.0005% by weight, wherein the tripeptide has a specific amino acid sequence obtained by specifically decomposing a collagen component or gelatin component derived from bone, skin or tendon (or sinew) of bovine or pig, or fish skin with a collagenase. However, as described above, a collagen or gelatin component derived from an animal is insufficient in safety.

Further, Japanese Patent Application Laid-Open No. 503873/1996 (JP-8-503873A) discloses use of a collagen as a carrier such as an excipient, a diluent or others. Japanese Patent Application Laid-Open No. 55263/2003 (JP-2003-55263A) discloses a composition for filling a soft capsule, containing a collagen and a collagen-derived substance as a carrier. Furthermore, a collagen is also utilized as an additive for enhancing a viscosity of a water phase (or an aqueous phase) in an oil-in-water type (“The Latest Pharmaceutics (sixth edition)”, Hirokawa Publishing Co., page 251, 1992). In general, a collagen derived from bovine or pig is often used as a raw material also in the field of the pharmaceutical preparation such as a medical supply. Among them, a bovine-derived collagen is in heavy usage because of excellent physicochemical properties and less smell.

The collagen is particularly rich in proline (Pro) and glycine (Gly). These two amino acid residues are important to form a stable triple helical structure of the collagen. In addition, the collagen is a naturally occurring fibrous material as described above, and is responsive to externally applied physical or chemical conditions such as temperature. Through the use of such collagen properties, a collagen or a heat-denatured product thereof (or a gelatin) has been utilized as various industrial materials.

For example, Japanese Patent Application Laid-Open No. 176472/2003 (JP-2003-176472A) discloses an adhesive agent composition as an adhesive agent suitable for adhesion between a non-porous base material (such as a polyolefin or a glass) and a paper, wherein the adhesive agent composition comprises a protein-series adhesive agent such as a glue or a gelatin, and at least one member selected from the group consisting of a rosin, a rosin derivative, a terpene and a terpene derivative mixed to the protein-series adhesive agent. Moreover, Japanese Patent Application Laid-Open No. 171521/1999 (JP-11-171521A) discloses a method for inhibiting leak out of filtration in an activated carbon used for decoloration and deodorization of a surfactant with the use of a collagen, which comprises treating the activated carbon with the collagen to agglutinate and link the activated carbons.

Moreover, in order to impart texture, external appearance, touch, function and others of a natural material such as human skin or leather to a coating composition containing a collagen, for example, Japanese Patent Application Laid-Open No. 60546/1996 (JP-8-60546A) discloses a collagen-containing paste for a fiber product such as a woven fabric and nonwoven fabric composed of a natural fiber, or a synthetic or semisynthetic fiber, the paste which contains a collagen and a macromolecular base material for the paste as essential components. Japanese Patent Application Laid-Open No. 59400/1993 (JP-5-59400A) discloses a paint composition containing a collagen, water, and a film-forming component. Japanese Patent Application Laid-Open No. 304960/1995 (JP-7-304960A) discloses a water-collagen powder dispersion as an additive for an aqueous fiber-treating agent, an aqueous finishing agent, or an aqueous paint. Japanese Patent Application Laid-Open No. 70600/1995 (JP-7-70600A) discloses a collagen powder which is used with mixing a paint, a treating agent for an artificial or synthetic leather, or a fiber-treating agent is mixed in use. Thus, although the collagen has been used as various industrial materials, because of the risks already pointed out in a mammal-derived collagen, the collagen is insufficient in safety in some industrial materials.

### SUMMARY OF THE INVENTION

It is therefore an object of the present invention to provide a bioapplicable material (or a bioapplicable composition, for example, a biomaterial or biocompatible material, a cosmetic preparation, a food composition, a pharmaceutical preparation composition (or an additive for a pharmaceutical preparation)) having a high safety, a high bioaffinity and biocompatibility.

It is another object of the present invention to provide a bioapplicable material (or a bioapplicable composition) or a film-forming material (or composition) having excellent collagen-like properties, in addition, being free from a risk of an infection of a pathogenic organism or a risk of a transmission of a causative factor and having a high safety.

A still another object of the present invention is to provide a bioapplicable material (or a biocompatible material) which is free from a risk of an undesired side effect as well as is degradable and sorbable (or absorbable or resorbable) in a living body.

It is a further object of the present invention to provide a cosmetic preparation inexpensively which is high in moisturizing property and stability, and is free from generation of an offensive smell.

It is a still further object of the present invention to provide a food composition which has biodegradability, and can enhance sorbability (or absorbability).

A further object of the present invention is to provide a pharmaceutical preparation composition (or an additive for a pharmaceutical preparation) useful for imparting sustained releasability.

A still further object of the present invention is to provide a film-forming material (or composition) which has a high adhesiveness to a base, an excellent biodegradability, and less environmental load.

The inventors of the present invention made intensive studies to achieve the above objects and finally found that a specific synthetic polypeptide was suitable for application to a living body since the polypeptide was free from a risk of an infection by a pathogenic organism or a risk of a transmission of a causative factor, and formed a collagen-like three-dimensional structure and tissue structure so as to have collagen-like (collagenous) properties; and that such a polypeptide had a high adhesiveness to a base (or a substrate) and was suitable for film-forming. The present invention was accomplished based on the above findings.

That is, the bioapplicable material or film-forming material of the present invention is a material or composition containing a polypeptide, wherein the polypeptide comprises a specific synthetic polypeptide. Incidentally, the material of the present invention may contain the synthetic polypeptide as at least a constituent component, or may contain the synthetic polypeptide as a main constituent component. Moreover, the material of the present invention may comprise the synthetic polypeptide alone, or may be a composition containing the synthetic polypeptide and other component(s) (for example, a carrier or base material component, an effective ingredient or an active ingredient, and an additive).

In the material or composition, the specific synthetic polypeptide has at least an amino acid sequence represented by the formula: Pro-Y-Gly (wherein Y represents Pro or Hyp), and can be form a collagen-like (or collagenous) structure.

The synthetic polypeptide may be at least one polypeptide selected from the group consisting of the following polypeptides (I) and (II):

(I) a polypeptide comprising peptide units represented by the following formulae (1) to (3):
[—(OC—(CH₂)ₘ—CO)ₚ-(Pro-Y-Gly)ₙ-]ₐ (1)
[—(OC—(CH₂)ₘ—CO)_{q}-(Z)ᵣ-]_{b} (2)
[—HN—R—NH—]_{c} (3)

wherein “m” denotes an integer of 1 to 18, “p” and “q” are the same or different, each representing 0 or 1, Y represents Pro or Hyp, and “n” denotes an integer of 1 to 20; Z represents a peptide chain comprising 1 to 10 amino acid residue(s), “r” denotes an integer of 1 to 20, and R represents a straight or branched chain alkylene group; the proportion (molar ratio) of “a” relative to “b” [(a)/(b)] is 100/0 to 30/70,

when p=1 and q=0, c=a,

when p=0 and q=1, c=b,

when p=1 and q=1, c=a+b, and

when p=0 and q=0, c=0; and

(II) a polypeptide comprising a peptide unit having an amino acid sequence represented by the following formula (4) and a peptide unit having an amino acid sequence represented by the following formula (5):
-Pro-Y-Gly- (4)

wherein Y has the same meaning as defined above;
-Pro-V-Gly-W-Ala-Gly- (5)

wherein V represents Gln, Asn, Leu, Ile, Val or Ala, and W represents Ile or Leu.

In the polypeptide (I), “m” may denote an integer of 2 to 12, “n” may denote an integer of 2 to 15, Z may represent an amino acid or peptide chain comprising 1 to 10 amino acid residue(s) selected from the group consisting of Gly, Sar, Ser, Glu, Asp, Lys, H is, Ala, Val, Leu, Arg, Pro, Tyr and Ile, “r” may denote an integer of 1 to 10, and R may be a C₂₋₁₂alkylene group. Moreover, the polypeptide (I) may comprise at least one member selected from the group consisting of the following polypeptides (i) to (iii):

(i) a polypeptide containing a unit represented by (Pro-Pro-Gly)ₙ,

(ii) a polypeptide containing a unit represented by (Pro-Hyp-Gly)ₙ, and

(iii) a polypeptide containing a unit represented by (Pro-Pro-Gly)ₙ₁ and a unit represented by (Pro-Hyp-Gly)ₙ₂.

Incidentally, in the polypeptides (i) to (iii), each of “n”, “n1” and “n2” represents a repeating number of each unit, and “n1” plus “n2” is “n”.

Further, in the polypeptide (II), the proportion (molar ratio) of the peptide unit (4) relative to the peptide unit (5) ((4)/(5)) may be about 99/1 to 30/70. Moreover, the polypeptide (II) may be a polypeptide in which (iv) Y represents Hyp, V represents one residue selected from the group consisting of Gln, Asn, Leu, Ile, Val and Ala, W represents Ile or Leu; or may be a polypeptide in which (v) Y represents Pro, V represents one residue selected from the group consisting of Gln, Asn, Leu, Ile, Val and Ala, and W represents Ile or Leu.

The synthetic polypeptide usually shows positive Cotton effect at a wavelength in range of 220 to 230 nm and negative Cotton effect at a wavelength in range of 195 to 205 nm in a circular dichroism spectrum. This fact indicates that at least part (part or whole) of the polypeptide forms a triple helical structure. The synthetic polypeptide may show a peak of the molecular weight in the range from 5×10³ to 500×10⁴ in the molecular weight distribution.

Furthermore, the polypeptide may be a biodegradable polypeptide, which is degradable and sorbable (or absorbable) in a living body. That is, the polypeptide may be degradable with a collagenase.

Such a synthetic polypeptide has a high biocompatibility, and is useful for a biomaterial (or a medical material) or a biocompatible material, a component of a pharmaceutical preparation (e.g., a medical pharmaceutical preparation), a blending component of an animal feeding stuff or a food. Moreover, since the synthetic polypeptide has not only biocompatibility but also a high moisturizing property and stability, the polypeptide is also useful for a cosmetic preparation (or a cosmetic composition).

The biomaterial (biocompatible material) or the medical material may include, for example, a coating material (or coating agent), an implant material (or implant agent), a hemostatic material (or hemostatic agent), an antiadhesive material (or antiadhesive agent), an adhesive material (or adhesive agent), a tube member, and a membrane material. Moreover, the biomaterial or medical material may comprise the polypeptide alone, or may comprise the polypeptide as a main constituent component. For example, the biomaterial or medical material may comprise a base, and the polypeptide applied on at least a surface of the base. The base may be degradable in a living body, that is, the base may have biodegradability.

The cosmetic preparation may be a powdery cosmetic preparation, a solid or semisolid cosmetic preparation, or a liquid cosmetic preparation. Moreover, the pharmaceutical preparation composition may be any of dosage forms of a solid pharmaceutical preparation, a liquid pharmaceutical preparation, or a semisolid pharmaceutical preparation. Further, the pharmaceutical preparation composition may be a sustained release pharmaceutical preparation. The food composition may be in the form of a powder, a solid, a semisolid, or a liquid. Furthermore, the food composition may be a food or an animal feeding stuff.

Since the synthetic polypeptide is excellent in adhesiveness to a base, the polypeptide is useful as a film-forming material (or composition) for applying the base, for example, as a coating agent or an adhesive agent.

Incidentally, in this specification, the term “bioapplicable material” (or bioapplicable composition) means a material suitable for application to a living body, e.g., animals such as human being as well as various non-human animals (e.g., reptiles, birds, fish, and mammals), and includes not only a variety of biomaterials or biocompatible materials, but also pharmaceutical preparation compositions to be administrated in a living body, cosmetic preparations for applying to living skin (or outer skin), hair, and others, food compositions for applying to a living body through buccal cavity and alimentary canal, and others.

In the present specification, amino acid residues are abbreviated to the following condensation codes.

Ala: L-alanine residue

Arg: L-arginine residue

Asn: L-asparagine residue

Asp: L-aspartic acid residue

Cys: L-cysteine residue

Gln: L-glutamine residue

Glu: L-glutamic acid residue

Gly: glycin residue

H is: L-histidine residue

Hyp: L-hydroxyproline residue

Ile: L-isoleucine residue

Leu: L-leucine residue

Lys: L-lysine residue

Met: L-methionine residue

Phe: L-phenylalanine residue

Pro: L-proline residue

Sar: sarcosine residue

Ser: L-serine residue

Thr: L-threonine residue

Trp: L-tryptophan residue

Tyr: L-tyrosine residue

Val: L-valine residue

Moreover, in this specification, amino acid sequences of peptide chains are represented in accordance with the conventional expression that N-terminus and C-terminus in an amino acid residue are drawn at the left and the right sides, respectively.

### BRIEF DESCRIPTION OF DRAWINGS

FIG. 1 is a scanning electron microphotograph representing a film obtained in Production Example 3.

### DETAILED DESCRIPTION OF THE INVENTION

The material of the present invention, that is, the bioapplicable material (or bioapplicable composition, for example, a biomaterial or a biocompatible material, a cosmetic preparation, a food composition, a pharmaceutical preparation composition (e.g., a medical pharmaceutical preparation composition or a medical additive)) or a film-forming material (or composition), comprises a specific synthetic polypeptide forming a collagen-like structure. The material may comprise the synthetic polypeptide alone, or may comprise the synthetic polypeptide and other component(s).

[Synthetic Polypeptide]

The synthetic polypeptide has at least an amino acid sequence represented by the formula: Pro-Y-Gly (wherein Y represents Pro or Hyp). Since the amino acid sequence contributes to stability of the triple helical structure, as the polypeptide, various polypeptides may be used as long as the polypeptide can form a collagen tissue (collagenous tissue) or collagen-like structure. Such a polypeptide may include the polypeptide (I) and the polypeptide (II).

In the polypeptide (I), it is necessary that the constitutive peptide unit (1) [—(OC—(CH₂)ₘ—CO)ₚ-(Pro-Y-Gly)ₙ-] contains a repeated sequence Pro-Y-Gly. In the case where the repeating number of the sequence Pro-Y-Gly is small, the triple helical structure deteriorates in stability. In the case where the repeating number is too large, it becomes difficult to synthesize such a peptide. Therefore, the repeating number “n” is about 1 to 20, preferably about 2 to 15 (e.g., about 3 to 15), and more preferably about 5 to 15.

In the formula (1), the residue “Y” may be either Pro or Hyp. In view of stability of the triple helical structure, the residue “Y” is more preferably Hyp. Incidentally, Hyp is usually 4Hyp (e.g., trans-4-hydroxy-L-proline) residue.

Further, the repeating number “m” of methylene chain (CH₂) is not particularly limited to a specific one as long as the physical and biological properties of the polypeptide are not deteriorated. The number “m” is usually about 1 to 18, preferably about 2 to 12, and more preferably about 2 to 10 (particularly about 2 to 6). The number “p” is 0 or 1.

In the peptide unit (2) [—(OC—(CH₂)ₘ—CO)_{q}-(Z)ᵣ-] “Z” represents a peptide chain of an arbitrary sequence comprising 1 to 10 amino acid residue(s). The sequence “Z” may be any sequence as far as the physical and biological properties of the obtained polypeptide is not deteriorated. In order that the polypeptide exerts useful physical and biological properties, for example, the peptide chain “Z” usually has a peptide chain comprising 1 to 10 amino acid residue(s) selected from the group consisting of Gly, Sar, Ser, Glu, Asp, Lys, H is, Ala, Val, Leu, Arg, Pro, Tyr, and Ile (that is, an amino acid residue selected from these amino acids, or a peptide chain comprising 2 to 10 amino acid residues selected from these amino acids), particularly, a peptide chain comprising 1 to 10 amino acid residue(s) selected from the group consisting of Gly, Sar, Ser, Glu, Asp, Lys, Arg, Pro, and Val in many cases. The peptide chain “Z” preferably contains Gly, Sar, Ser, Glu, Asp, Lys, or a sequence represented by Arg-Gly-Asp, Tyr-Ile-Gly-Ser-Arg, Ile-Lys-Val-Ala-Val, Val-Pro-Gly-Val-Gly, Asp-Gly-Glu-Ala, Gly-Ile-Ala-Gly, His-Ala-Val, Glu-Arg-Leu-Glu, Lys-Asp-Pro-Lys-Arg-Leu, or Arg-Ser-Arg-Lys.

The repeating number “r” of the peptide chain “Z” is not particularly limited to a specific one as long as the obtained polypeptide exerts physical and biological properties. In the case where the repeating number “r” is too large, it is difficult to synthesize the peptide, and the physical properties of the obtained peptide tend to change. Therefore, the repeating number “r” is usually about 1 to 20, preferably about 1 to 10, and more preferably about 1 to 5.

The repeating number “m” of the methylene chain (CH₂) is, as is the case with the formula (1), about 1 to 18, preferably about 2 to 12, and more preferably about 2 to 10 (particularly about 2 to 6). The number “q” is 0 or 1.

In the formulae (1) and (2), when at least one of “p” and “q” is 1, the polypeptide contains the unit [—HN—R—NH—] represented by the formula (3). In the unit represented by the formula (3), the straight or branched chain alkylene group represented by “R” may be any groups as long as the physical and biological properties of the polypeptide are not deteriorated. For example, the group “R” may include a C₁₋₁₈alkylene group such as methylene, ethylene, propylene, trimethylene, or tetramethylene. The alkylene group “R” may be a straight methylene chain (CH₂)ₛ (“s” denotes an integer of 1 to 18). The preferred “R” includes a C₂₋₁₂alkylene group (more preferably a C₂₋₁₀alkylene group, and particularly a C₂₋₆alkylene group).

The proportion (molar ratio) (a/b) of the peptide unit represented by the formula (1) relative to the peptide unit represented by the formula (2) is (a/b)=about 100/0 to 30/70, preferably about 100/0 to 40/60, and more preferably about 100/0 to 50/50 (e.g., about 95/5 to 50/50).

Further, the proportion of the unit represented by the formula (3) may be selected depending on the value “p” of the formula (1) and the value “q” of the formula (2). When p=1 and q=0, c=a; and when p=0 and q=1, c=b. Moreover, when p=1 and q=1, c=a+b; and when p=0 and q=0, c=0.

That is, the polypeptide (I) includes (a) a polypeptide comprising the repeating peptide unit [-(Pro-Y-Gly)ₙ-] (wherein p=0 in the formula (1)); (b) a polypeptide comprising a repeating unit containing the peptide unit [-(Pro-Y-Gly)ₙ-] (wherein p=0 in the formula (1)) and the peptide unit [-(Z)ᵣ-] (wherein q=0 in the formula (2)) in a proportion of a/b (% by mol); (c) a polypeptide comprising a repeating unit containing the peptide unit [—(OC—(CH₂)ₘ—CO)-(Pro-Y-Gly)ₙ-] (wherein p=1 in the formula (1)) and the unit [—HN—R—NH—] represented by the formula (3) in a molar ratio of 1/1; and (d) a polypeptide comprising a repeating unit containing the peptide unit [—(OC—(CH₂)ₘ—CO)-(Pro-Y-Gly)ₙ-] (wherein p=1 in the formula (1)), the peptide unit [—(OC—(CH₂)ₘ—CO)-(Z)ᵣ-] (wherein q=1 in the formula (2)), and the unit [—HN—R—NH—] represented by the formula (3) in a molar ratio of a/b/(a+b).

Among the polypeptides (I), the particularly preferred one includes at least one selected from the group consisting of (i) a polypeptide containing a unit represented by (Pro-Pro-Gly)ₙ, (ii) a polypeptide containing a unit represented by (Pro-Hyp-Gly)ₙ, and (iii) polypeptide containing a unit represented by (Pro-Pro-Gly)ₙ₁ and a unit represented by (Pro-Hyp-Gly)ₙ₂. In the polypeptides (i) to (iii), each of “n1” and “n2” represents a repeating number of each unit, and “n1” plus “n2” is “n”.

In the polypeptide containing both units -Pro-Pro-Gly- and -Pro-Hyp-Gly-, the proportion (molar ratio) of the unit (Pro-Pro-Gly)ₙ₁ relative to the unit (Pro-Hyp-Gly)ₙ₂ may be about 0.1/99.9 to 99.9/0.1, preferably about 0.5/99.5 to 90/10, and more preferably about 1/99 to 80/20 (e.g., about 5/95 to 60/40).

On the other hand, it is necessary that the polypeptide (II) contains the peptide unit (4) having an amino acid sequence represented by -Pro-Y-Gly-. Since the sequence represented by -Pro-Y-Gly- contributes to stability of the triple helical structure, the low proportion of the sequence brings about deterioration in stability of the triple helical structure.

Further, in view of stability of the triple helical structure, the unit (4) may form a repeated structure (an oligo or polypeptide unit structure) represented by -(Pro-Y-Gly)_{d}- in the polypeptide. The repeating number “d” of the sequence is, for example, about 2 to 5000, preferably about 2 to 4000, and more preferably about 3 to 3000. The residue “Y” may be either Pro or Hyp. In the same manner as the above, Hyp [usually, 4Hyp (e.g., trans-4-hydroxy-L-proline) residue] is more preferred in view of stability of the triple helical structure.

Moreover, it is useful that the polypeptide (II) in the present invention contains a peptide unit (5) having an amino acid sequence represented by -Pro-V-Gly-W-Ala-Gly-. In the case where the polypeptide (II) does not contain this sequence or contains this sequence in too low proportion, the polypeptide decreases degradability with a collagenase. On the other hand, too high proportion of the sequence brings about deterioration in stability of the triple helical structure.

The residue “V” may be Gln, Asn, Leu, Ile, Val or Ala, and is preferably Gln, Asn, Leu, Val, or Ala. In particular, Gln or Leu is more preferred. The residue “W” may be either Ile or Leu, and is more preferably Ile.

With respect to the combination of the residues “V” and “W”, for example, the peptide may include a peptide in which the residue “V” is Gln, Asn, Leu, Ile, Val or Ala (e.g., Gln or Leu), the residue “W” is Ile; a peptide in which the residue “V” is Gln, Asn, Leu, Ile, Val or Ala (e.g., Gln or Leu), and the residue “W” is Leu; and others.

With respect to the combination of the residues “Y”, “V” and “W”, the peptide may include a peptide in which the residue “Y” is Hyp, the residue “V” is Gln, Asn, Leu, Ile, Val or Ala (e.g., Gln or Leu), and the residue “W” is Ile or Leu; a peptide in which the residue “Y” is Pro, the residue “V” is Gln, Asn, Leu, Ile, Val or Ala (e.g., Gln or Leu), the residue “W” is Ile or Leu; and others.

Further, unless the physical and biological properties of the obtained polypeptide are deteriorated, the polypeptide may contain other amino acid residue(s) or peptide residue(s) (unit(s)). Other amino acid residue or peptide chain may include a peptide chain represented by -(Z)ᵣ- of the peptide unit (2), and the like. That is, in order that the polypeptide exerts useful physical and biological properties, for example, the polypeptide often has a peptide chain comprising 1 to 10 amino acid residue(s) selected from the group consisting of Gly, Sar, Ser, Glu, Asp, Lys, H is, Ala, Val, Leu, Arg, Pro, Tyr, and Ile (that is, an amino acid residue selected from these amino acids, or a peptide chain comprising 2 to 10 amino acid residues selected from these amino acids), particularly, a peptide chain comprising 1 to 10 amino acid residue(s) selected from the group consisting of Gly, Sar, Ser, Glu, Asp, Lys, Arg, Pro, and Val. More specifically, for example, it is preferred to contain an amino acid residue or peptide residue represented by Gly, Sar, Ser, Glu, Asp, Lys, Arg-Gly-Asp, Tyr-Ile-Gly-Ser-Arg, Ile-Lys-Val-Ala-Val, Val-Pro-Gly-Val-Gly, Asp-Gly-Glu-Ala, Gly-Ile-Ala-Gly, His-Ala-Val, Glu-Arg-Leu-Glu, Lys-Asp-Pro-Lys-Arg-Leu, or Arg-Ser-Arg-Lys.

In the polypeptide (II), the proportion (molar ratio) of the peptide unit (4) relative to the peptide unit (5) is (4)/(5)=about 99/1 to 30/70, preferably about 98/2 to 40/60, and more preferably about 95/5 to 50/50.

The proportion (molar ratio) of the total amount of the peptide units (4) and (5) relative to other peptide unit(s) [the former/the latter] is about 100/0 to 50/50, preferably about 100/0 to 60/40, and more preferably about 100/0 to 70/30 (e.g., about 95/5 to 70/30).

Such polypeptides (I) and (II) take a linear polypeptide formation without forming a ring such as a six-membered ring by cyclization, and is soluble in a solvent (for example, water, a hydrophilic solvent such as an alcohol such as ethanol or propanol, a ketone such as acetone, a cyclic ether such as dioxane or tetrahydrofuran, a sulfoxide such as dimethyl sulfoxide, dimethylformamide, dimethylacetamide, or N-methylpyrrolidone, or a mixed solvent thereof). The polypeptide of the present invention shows, for example, a peak of the molecular weight in the range from about 5×10³ to 500×10⁴, preferably about 1×10⁴ to 300×10⁴, preferably about 3×10⁴ to 200×10⁴, and more preferably about 5×10⁴ to 100×10⁴ in the molecular weight distribution. Incidentally, the molecular weight (or the peak of the molecular weight) of the polypeptide is determined in terms of a globular protein by means of an aqueous gel permeation chromatography (GPC).

Further, these polypeptides show positive Cotton effect at a wavelength in a range of 220 to 230 nm and negative Cotton effect at a wavelength in a range of 195 to 205 nm in circular dichroism spectra. At least one part (that is, part or whole) of the polypeptide is, accordingly, capable of forming a triple helical structure, and the polypeptide forms a collagenous (collagen-like) structure. Incidentally, Cotton effect means a phenomenon caused by difference between an absorption coefficient relative to a right circularly polarized light and that relative to a left at a specific wavelength in an optical rotatory substance.

These polypeptides are capable of forming a collagen tissue (or a collagenous tissue). The polypeptide chains having the above-mentioned triple helical structure can self-assemble to form a fibril having a length of several nanometers to several tens nanometers. Further, these fibrils can be arranged to form a fiber structure having a length of several nanometers to several tens nanometers. These can be observed by a transmission electron microscope, a scanning electron microscope, or an atomic force microscope.

The polypeptides (I) and (II) may have biodegradability, particularly degradability in a living body. Such a biodegradable polypeptide is degradable with a collagenase. In particular, the polypeptide (II) shows a high biodegradability.

These polypeptides may be a physiologically or pharmacologically acceptable salt, and for example, may be a salt with a salifiable compound such as an inorganic acid (e.g., a hydrochloric acid, a sulfuric acid, and a phosphoric acid), an organic acid (e.g., acetic acid, trifluoroacetic acid, lactic acid, tartaric acid, maleic acid, fumaric acid, oxalic acid, malic acid, citric acid, oleic acid, and palmitic acid), a metal (e.g., an alkali metal such as sodium or potassium, an alkaline earth metal such as calcium, and aluminum), or an organic base (e.g., trimethylamine, triethylamine, t-butylamine, benzylamine, diethanolamine, dicyclohexylamine, and arginine). These salifiable compounds may be used singly or in combination. These salts may be obtained by a conventional salt-forming reaction.

These polypeptides (I) and (II) may be obtained by a conventional method which comprises subjecting an amino acid or a peptide fragment (or segment) to a condensation reaction, and is not particularly limited to a specific one as long as the polypeptide finally contains the above-mentioned units. For example, the polypeptide may be obtained by utilizing a condensation reaction between constituent amino acids, or a condensation reaction between a peptide segment and an amino acid. These polypeptides are preferably obtained by a method which comprises preparing a peptide component such as a peptide having the above-mentioned amino acid sequence(s), or a derivative thereof in advance, and condensing the peptide component.

In the method which comprises condensing the peptide component prepared in advance, the peptide chain of the peptide component can be synthesized in accordance with a conventional peptide synthesis method. Peptides may, for example, be prepared based on a solid-phase synthesis method or a liquid-phase synthesis method, and the solid-phase synthesis method is operationally convenient [for example, see “Zoku Seikagaku Jikken Kouza 2, Tanpakushitsu no Kagaku (Supplemental Handbook of Biochemical Experiments, Chemistry of Protein) (the second volume)” edited by The Japanese Biochemical Society (issued by Tokyo Kagaku Dozin Co., Ltd., May 20, 1987), pp. 641 to 694]. For the peptide synthesis, a conventional manner may be utilized, and the manner may include, for example, a coupling method using a condensing agent, an active esterification method (e.g., a phenyl ester such as p-nitrophenyl ester (ONp) and pentafluorophenyl ester (Opfp), an N-hydroxydicarboxylic imide ester such as N-hydroxysuccinimide ester (ONSu), and 1-hydroxybenzotriazole ester (Obt)), a mixed acid anhydride method, an azide method, and others. In the preferred manner, at least a condensing agent (preferably a condensing agent as mentioned below, in particular a combination of a condensing agent as mentioned below with a condensing auxiliary as mentioned below) may be practically used.

Furthermore, in the peptide synthesis, protection of an amino group, a carboxyl group, and other functional group (e.g., a guanidino group, an imidazolyl group, a mercapto group, a hydroxyl group, and an w-carboxyl group) with a protective group, and elimination or removal of the protective group with a catalytic reduction or a strong acid treatment (e.g., anhydrous hydrogen fluoride, trifluoromethanesulfonic acid, and trifluoroacetic acid) are repeatedly conducted depending on a species of amino acids or peptide fragments. For example, as a protective group for an amino group, there may be utilized benzyloxycarbonyl group (Z), p-methoxybenzyloxycarbonyl group (Z(OMe)), 9-fluorenylmethoxycarbonyl group (Fmoc), t-butoxycarbonyl group (Boc), 3-nitro-2-pyridinesulfenyl group (Npys), and the other groups. As a protective group for a carboxyl group, there may be utilized benzyloxy group (OBzl), phenacyloxy group (OPac), t-butoxy group (OBu), methoxy group (OMe), ethoxy group (OEt), and the other groups. Incidentally, an automatic synthesis apparatus may be utilized for the peptide synthesis.

More specifically, the preparation of the peptide chain with the solid-phase synthesis method may be carried out in accordance with a conventional manner. As a solid-phase resin (or a carrier), there may be utilized a polymer insoluble to a reaction solvent, for example, a styrene-divinylbenzene copolymer (e.g., a chloromethylated resin, a hydroxymethyl resin, a hydroxymethylphenylacetamidemethyl resin, and a 4-methylbenzhydrylamine resin).

In the solid-phase synthesis method, a peptide can be usually produced by the following steps: a step forming a peptide chain corresponding to an objective peptide, which comprises operations (i) to (iii) mentioned below, and a step comprising (iv) detaching the peptide chain from the polymer (resin) and eliminating the protective group(s) from the protected functional group(s) to obtain the objective peptide, and purifying the resulting peptide. The peptide chain-forming step comprises (i) bonding an amino acid or peptide fragment to the above polymer (resin) from C-terminal to N-terminal of the objective peptide, in which the amino acid or peptide fragment has a free α-COOH group and a functional group(s) (e.g., at least an α-amino group of the N-terminal) protected with a protective group(s), (ii) eliminating the protective group from the α-amino group for forming a peptide bond among the bonded amino acid or peptide fragment, and (iii) sequentially repeating the above bonding operation and the eliminating operation to elongate the peptide chain for the formation of the object peptide. In the operation (i) for bonding the amino acid or peptide fragment, an amino acid which is corresponding to the C-terminal of the peptide chain and has a free α-COOH group, and in which at least the N-terminal is protected with a protective group (for example, an Fmoc-amino acid, a Boc-amino acid) is used. Incidentally, from the viewpoint of inhibiting a side reaction, detachment of the peptide chain from the polymer is preferably carried out concurrently with elimination of the protective group with the use of trifluoroacetic acid. Moreover, the resulting peptide may be purified by utilizing a separation and purification means (e.g., a reversed phase liquid chromatography, and a gel-permeation chromatography).

The polypeptide (I) is, for example, prepared by condensing at least (A) a peptide represented by the following formula (1a) or a derivative thereof.
X-(Pro-Y-Gly)ₙ-OH (1a)

In the formula, “X” represents H or HOOC—(CH₂)ₘ—CO— (“m” has the same meaning as defined above), “Y” and “n” have the same meaning as defined above.

The polypeptide may be prepared by co-condensing (A) a peptide or a derivative thereof represented by the above formula (1a) with (B) a peptide or a derivative thereof represented by the following formula (2a):
X-(Z)ᵣ-OH (2a)

wherein X represents H or HOOC—(CH₂)ₘ—CO— (“m” has the same meaning as defined above), “Z” and “r” have the same meanings as defined above.

Incidentally, as the compound in which the above group “X” is HOOC—(CH₂)ₘ—CO—, there may be mentioned, for example, a C₃₋₂₀ aliphatic dicarboxylic acid such as malonic acid, succinic acid, glutaric acid, adipic acid, pimelic acid, suberic acid, azelaic acid or sebacic acid, or an acid anhydride thereof. These compounds may be used singly or in combination. These compounds may be also subjected to a reaction according to a conventional amide bond-forming method (for example, a reaction using a tertiary amine described later as a catalyst) or the above-mentioned peptide synthesis method to obtain the compounds represented by the above formulae (1a) and (2a).

The ratio of the peptide or derivative thereof (A) relative to the peptide or derivative thereof (B) [the former (A)/the latter (B)] is, for example, about 100/0 to 30/70 (mol %), preferably about 100/0 to 40/60 (mol %), and more preferably about 100/0 to 50/50 (e.g., about 95/5 to 50/50) (mol %).

Further, in the above formulae (1a) and/or (2a), if the group “X” is HOOC—(CH₂)ₘ—CO— (“m” has the same meaning as defined above), at least one of the peptide or derivative thereof (A) and the peptide or derivative thereof (B) is subjected to a co-condensation reaction with the compound (C) represented by the following formula (3a) for forming an amide group. If the group “X” is H, it is unnecessary to use the compound (C).
H₂N—R—NH₂ (3a)

“R” has the same meaning defined above.

As the compound represented by the above formula (3a), there may be exemplified a diamine corresponding to the above formula (3), e.g., a C₁₋₁₈alkylenediamine such as ethylenediamine, trimethylenediamine, propylenediamine, tetramethylenediamine or hexamethylenediamine, a polyalkylenepolyamine such as diethylenetriamine or hexamethylenetetramine, and others. These compounds may be used singly or in combination.

The amount of the diamine compound (C), in the case where the group “X” is HOOC—(CH₂)ₘ—CO— (“m” has the same meaning as defined above) in either the peptide or derivative thereof (A) or (B), the amount of the diamine compound (C) may be substantially 1 mol (for example, about 0.95 to 1.05 mol) relative to 1 mol of the peptide or derivative thereof having such group.

In the preparation of the polypeptide (II), a method for reacting a peptide component at least containing a peptide having the amino acid sequence may include (a) a method which comprises condensing a peptide component at least containing a peptide having the both amino acid sequences represented by the formulae (4) and (5) (that is, a peptide having both a peptide unit having an amino acid sequence represented by the formula (4) and a peptide unit having an amino acid sequence represented by the formula (5)); and (b) a method which comprises condensing a peptide component at least containing a peptide having an amino acid sequence represented by the formula (4) and a peptide having an amino acid sequence represented by the formula (5).

In the former method (a), the peptide having the both amino acid sequences represented by the formulae (1) and (2) may be used singly or in combination. Moreover, in this method, as a peptide component, other peptide(s) may be used in addition to the above-mentioned peptide, depending on an object polypeptide. Other peptide(s) may include, for example, a peptide having an amino acid sequence represented by the formula (1), a peptide having an amino acid sequence represented by the formula (2), and in addition a peptide containing the above-mentioned other amino acid residue(s) or peptide chain(s), and others. These other peptides may be used singly or in combination. Incidentally, in the method, the proportion of the unit (1) or (2) may be easily adjusted by co-condensing a peptide having an amino acid sequence represented by the formula (1) or (2).

Also in the latter method (b), each of a peptide (oligo or polypeptide unit) having an amino acid sequence represented by the formula (1), and a peptide having an amino acid sequence represented by the formula (2) may be used singly or in combination. Moreover, also in the method, as a peptide component, other peptide(s) may be used in addition to these peptides (1) and (2), depending on an object polypeptide. Examples of other peptide(s) may include a peptide containing the above-mentioned other amino acid residue(s) or peptide chain(s), and others. These other peptides may be used singly or in combination.

The condensation reaction of these peptide components is usually carried out in a solvent. The solvent may be capable of dissolving or suspending (partly or wholly dissolving) the peptide components, and there may be usually employed water and/or an organic solvent. Examples of the solvent may include water, an amide (e.g., dimethylformamide, dimethylacetamide, and hexamethylphosphoramide), a sulfoxide (e.g., dimethyl sulfoxide), a nitrogen-containing cyclic compound (e.g., N-methylpyrrolidone, and pyridine), a nitrile (e.g., acetonitrile), an ether (e.g., dioxane, and tetrahydrofuran), an alcohol (e.g., methyl alcohol, ethyl alcohol, and propyl alcohol), and a mixed solvent thereof. Among these solvents, water, dimethylformamide, or dimethyl sulfoxide is practically used.

The reaction of these peptide components may be usually carried out in the presence of at least a dehydrating agent (a dehydrating and condensing agent) or a condensing agent. The reaction with these peptide components in the presence of a dehydrating and condensing agent and a condensing auxiliary (synergist) smoothly produces a polypeptide with inhibiting dimerization or cyclization.

The dehydrating and condensing agent is not particularly limited to a specific one as far as the agent can conduct dehydration and condensation efficiently in the above-mentioned solvent. For example, the dehydrating and condensing agent (the dehydrating agent) may include a carbodiimide-series condensing agent [e.g., diisopropylcarbodiimide (DIPC), 1-ethyl-3-(3-dimethylaminopropyl)-carbodiimide (EDC=WSCI), 1-ethyl-3-(3-dimethylaminopropyl)-carbodiimide hydrochloride (WSCI.HCl), and dicyclohexylcarbodiimide (DCC)], a fluorophosphate-series condensing agent [e.g., O-(7-azabenzotriazol-1-yl)-1,1,3,3-tetramethyluronium hexafluorophosphate, O-benzotriazol-1-yl-N,N,N′,N′-tetramethyluronium hexafluorophosphate, benzotriazol-1-yl-oxy-tris-pyrrolidinophosphonium hexafluorophosphate, and a salt of benzotriazol-1-yl-tris(dimethylamino)phosphonium hexafluorophosphide (BOP)], diphenylphosphorylazide (DPPA), and others. The dehydrating and condensing agent(s) may be used singly, or used as a mixture in combination thereof. The preferred dehydrating and condensing agent includes a carbodiimide-series condensing agent [e.g., 1-ethyl-3-(3-dimethylaminopropyl)-carbodiimide, and 1-ethyl-3-(3-dimethylaminopropyl)-carbodiimide hydrochloride].

The condensing auxiliary is not particularly limited to a specific one as long as the condensing auxiliary can facilitate the reaction of the condensing agent. For example, there may be mentioned an N-hydroxypolycarboxylic imide [e.g., an N-hydroxydicarboxylic imide such as N-hydroxysuccinic imide (HONSu) or N-hydroxy-5-norbornene-2,3-dicarboxylic imide (HONB)]; an N-hydroxytriazole [e.g., an N-hydroxybenzotriazole such as 1-hydroxybenzotriazole (HOBt)]; a triazine such as 3-hydroxy-4-oxo-3,4-dihydro-1,2,3-benzotriazine (HOObt); ethyl ester of 2-hydroxyimino-2-cyanoacetic acid; and others. These condensing auxiliaries may be also used singly or in combination. The preferred condensing auxiliary includes an N-hydroxydicarboxylic imide [e.g., HONSu], an N-hydroxybenzotriazole or N-hydroxybenzotriazine [e.g., HOBt].

The dehydrating and condensing agent may be suitably used in combination with the condensing auxiliary. As a combination of the dehydrating and condensing agent with the condensing auxiliary, there may be mentioned, for example, DCC-HONSu (HOBt or HOObt), WSCI-HONSu (HOBt or HOObt), and other combinations.

The amount to be used of the dehydrating and condensing agent is, in a water-free solvent system, usually about 0.7 to 5 mol, preferably about 0.8 to 2.5 mol, and more preferably about 0.9 to 2.3 mol (e.g., about 1 to 2 mol) relative to 1 mol of the total molar amount of the peptide components (including the diamine compound). In a water-containing solvent (or an aqueous solvent) system, since the dehydrating and condensing agent may be deactivated by water, the amount to be used of the dehydrating and condensing agent is usually about 2 to 500 mol (e.g., about 2 to 50 mol), preferably about 5 to 250 mol (e.g., about 5 to 25 mol), and more preferably about 10 to 125 mol (e.g., about 10 to 20 mol) relative to 1 mol of a total molar amount of the peptide components. The amount to be used of the condensing auxiliary is, for example, about 0.5 to 5 mol, preferably about 0.7 to 2 mol, and more preferably about 0.8 to 1.5 mol relative to 1 mol of a total molar amount of the peptides irrespective of a kind or species of the solvent.

In the condensation reaction, the pH of the reaction system may be adjusted, or abase being inert for the reaction may be added to the system. The pH may be usually adjusted with an inorganic base [e.g., sodium hydroxide, potassium hydroxide, sodium carbonate, and sodium hydrogen carbonate], an organic base, an inorganic acid [e.g., hydrochloric acid], or an organic acid. The pH of the reaction mixture is usually adjusted to approximately neutral pH (pH=about 6 to 8). As the base being inert for the reaction, there may be exemplified a tertiary amine (e.g., a trialkylamine such as trimethylamine, triethylamine or diisopropylethylamine, and a heterocyclic tertiary amine such as N-methylmorpholine or pyridine), and others. The amount of the base may be usually selected within a range from one to two times as much as the total molar amount of the peptides.

The formation of a triple helical structure in the polypeptide can be usually proved by measuring circular dichroism spectra for a solution of the polypeptide. In particular, regarding circular dichroism spectra, it has been reported that a naturally-occurring collagen and peptide chain forming a triple helical structure distinctively shows positive Cotton effect at a wavelength in range of 220 to 230 nm and negative Cotton effect at a wavelength in range of 195 to 205 nm (J. Mol. Biol., Vol. 63 pp. 85 to 99, 1972).

Such a polypeptide is, different from a collagen derived from mammals, free from a risk of an infection or a transmission of a pathogenic organism or a causative factor [for example, a protein converted into a pathological protein (e.g., abnormal prion)]. Therefore, the above-mentioned polypeptide is high in safety.

Moreover, the synthetic polypeptide is capable of forming a collagen-like (or collagenous) tissue, and is also excellent in cytophilicity or biocompatibility. The synthetic polypeptide is therefore useful for various bioapplicable materials. The synthetic polypeptide is applicable to a biomaterial or biocompatible material (or medical material) as an artificial collagen. Further, the polypeptide has a high moisturizing property and high stability, and is useful for a component of a cosmetic preparation. Since the polypeptide is high in safety, excellent in stability, biodegradability, and biosorbability (or bioabsorbability) as described above, the polypeptide is useful for a component of a food composition (a food, as well as an animal feeding stuff for domestic animals, pets, fish, and others). Moreover, the polypeptide is capable of forming a collagen-like tissue and also suitable for a pharmaceutical preparation component (e.g., a component of a medical pharmaceutical preparation) such as an active ingredient, a carrier, or an additive. Further, the polypeptide is free from generation of an offensive smell derived from a living organism (e.g., animals, and fish). Therefore, the polypeptide is preferably used for a pharmaceutical preparation composition (e.g., a medical composition) as an artificial collagen.

Moreover, the polypeptide is excellent in film-formability or moldability (or formability) and is easy to form a desired shape. Further, due to high safety as described above, the polypeptide is usable for various industrial materials. In particular, even in applications in which the polypeptide contacts with a human body, or in which the infant may accidentally put the polypeptide into his/her mouth, the polypeptide can be used with a safe conscience. Furthermore, since the polypeptide is high in stability (heat stability) and has a high adhesiveness to a base, the polypeptide is useful for a film-forming composition (a coating agent or an adhesive agent). Incidentally, since the polypeptide (particularly the polypeptide (II)) is a collagen-like substance, the polypeptide is also excellent in biodegradability and has less environmental load.

[Bioapplicable Material (or Bioapplicable Composition)]

(Biomaterial (or Biocompatible Material) or Medical Material)

The polypeptide is excellent in film-formability or moldability (or formability) and is easy to form a desired shape. Therefore, the biomaterial or medical material may comprise the polypeptide alone. For example, the biomaterial may be used in various forms comprising the polypeptide, e.g., in a liquid form (e.g., a solution or a suspension), a particulate form, a two-dimensional form (e.g., a film or a sheet), and a three-dimensional form. A liquid polypeptide may be prepared as a mixture of a polypeptide with water, an organic solvent (e.g., dimethyl sulfoxide, hexafluoroisopropanol), or a mixed solvent thereof. A particulate polypeptide may be, for example, prepared by pulverizing a polypeptide or spray-drying a solution or suspension of a polypeptide. A sheet or film of the polypeptide may be obtained by casting a solution or suspension of the polypeptide on a releasable substrate (support) (e.g., a sheet made from a fluorine-containing resin (polytetrafluoroethylene)) and drying the cast substrate. Moreover, a fibrous substance is obtained by extruding a solution or suspension of the polypeptide through a nozzle in a solution containing a salt of high concentration or in a solvent incapable of dissolving the polypeptide. Further, a non-woven fabric may be obtained from the fibrous polypeptide with the use of a wet or dry paper production process. Furthermore, a gelatinous substance may be obtained by allowing to stand an aqueous solution or suspension of the polypeptide, or if necessary, with adding a crosslinking agent thereto. Further, a sponge-like porous substance may be obtained by lyophilizing the resultant gelatinous substance. Furthermore, a porous substance can be also obtained by stirring the aqueous solution or suspension of the polypeptide to foam, and drying.

Incidentally, not only the gelatinous substance or porous substance, but also the particulate, fibrous, sheet- or film-like polypeptide, if necessary, may be also crosslinked with a crosslinking agent. The crosslinking agent may include, for example, a physiologically acceptable crosslinking agent such as a dialdehyde compound (e.g., glyoxal, glutaraldehyde, and succinaldehyde), a dextrandialdehyde, or an aldehyde starch. The proportion of the crosslinking agent may be about 1 to 20 parts by weight, and preferably about 1 to 10 parts by weight relative to 100 parts by weight of the polypeptide.

The biomaterial or medical material of the present invention may be also a composition containing the polypeptide. The composition may contain, for example, an active ingredient (a physiologically or pharmacologically acceptable active ingredient), a carrier, an additive, and others. The active ingredient may include, for example, a germicide (or microbicide) or a disinfectant, an antiinflammatory agent, an antiphlogistic and analgesic, an antipruritic, an antiulcer agent, an antiallergic agent, a vicucide, an antifungal agent, an antibiotic, an emollient, a therapeutic agent for bedsore (or decubitus) and skin, a vitamin preparation, and a herbal preparation. Moreover, as the active ingredient, a hemostatic component (e.g., a fibrin for stanching the bleeding by a blood coagulation factor), a polypeptide or a salt thereof having a cell proliferation and promoting action, a vascularization action, and/or a cell adhesion action (Japanese Patent Application Laid-Open No. 316581/1998 (JP-10-316581A)), and others may be contained. These components may be used singly or in combination.

As the carrier, various physiologically acceptable carriers may be used depending on the dosage form of the biomaterial (e.g., a solid preparation, a semisolid preparation, and a liquid preparation). For example, the carrier for the solid preparation may include a binder [for example, a component constituting the following organic base material, and in addition a cellulose derivative (e.g., a cellulose ether such as a methylcellulose, an ethylcellulose, a carboxymethylcellulose sodium, a hydroxyethylcellulose, a hydroxypropylcellulose, a hydroxypropylmethylcellulose, or a low-substituted hydroxypropylcellulose, and a cellulose ester such as a cellulose acetate), a polyvinylpyrrolidone, a polyvinyl alcohol, an acrylic polymer (e.g., a poly(sodium acrylate)), and a polysaccharide (e.g., a gum Arabic (powder), a pullulan, a pregelatinized starch, a sodium alginate, and a guar gum)], an excipient (e.g., lactose, sucrose, mannitol, a corn starch, a crystalline cellulose, and light silicic anhydride), and a disintegrant (e.g., a crosslinked povidone, a crosslinked carmellose sodium, and a corn starch).

Examples of the carrier for the semisolid preparation may include a base material (e.g., a vaseline, a liquid paraffin, a paraffin, Plastibase, a lanolin, a vegetable oil, a wax, a silicone, and a polyethylene glycol), and may be an aqueous (or water-containing) base material or a gel base material.

For the liquid preparation, the carrier may include, for example, water, an alcohol (e.g., ethanol), ethylene glycol, propylene glycol, a polyethylene glycol-polypropylene glycol copolymer, a fat and oil (e.g., isopropyl myristate, a corn oil, and an olive oil). To the liquid preparation may be added various additives, for example, a suspending agent [for example, a surfactant; a water-soluble polymer, e.g., a polyvinyl alcohol, a polyvinylpyrrolidone, and a water-soluble cellulose ether (e.g., a methylcellulose, a hydroxyethylcellulose, a hydroxypropylcellulose, and a carboxymethylcellulose sodium)], a buffer (e.g., a buffer solution such as a phosphate or a borate), a dissolution aid or a solubilizing agent (e.g., a polyoxyethylene hydrogenated castor oil, lecithin, a polyethylene glycol, ethanol, trisaminomethane, triethanolamine, sodium carbonate, and sodium citrate), a preservative (e.g., p-oxybenzoic acid ester, dehydroacetic acid, sorbic acid or a salt thereof, chlorobutanol, and benzyl alcohol), and an antioxidant (e.g. a sulfite salt, ascorbic acid, and α-tocopherol).

Incidentally, the surfactant in the suspending agent may include an anionic surfactant (e.g., an alkyl sulfate salt such as sodium lauryl sulfate; an alkyl ether sulfate salt such as a sodium alkyl ether sulfate, a triethanolamine alkyl ether sulfate; an acylmethyl taurine salt; an acylglutamate such as sodium acylglutamate; an amide ether sulfate; a sorbitan fatty acid ester such as sorbitan sesquioleic acid ester; a glycerin fatty acid ester such as glyceryl monostearate; and a polyoxyethylene glycerin fatty acid ester such as a polyoxyethylene glyceryl monostearate), and an ampholytic surfactant (e.g., an alkylacetic acid betaine, an amidoacetic acid betaine, and an imidazolinium betaine (an amine oxide-based semipolar surfactant)), a nonionic surfactant (e.g., a fatty acid alkanol amide such as lauric acid diethanolamide, or palm fatty acid diethanolamide; a polyoxyethylene alkyl ether such as a polyoxyethylene oleyl ether, or a polyoxyethylene octyl dodecyl ether; a polyoxyethylene glycerin fatty acid ester, a polyoxyethylene sorbitan fatty acid ester; a polyoxyethylene-polyoxypropylene block copolymer; and a polyoxyethylene hydrogenated castor oil ester), a cationic surfactant (e.g., an alkyltrimethylammonium chloride, a dialkyldimethylammonium chloride, a benzalkonium chloride, and benzethonium chloride), and others.

Incidentally, the carrier, the active ingredient and the additive may be used in the form of a salt. As such a salt, a physiologically or pharmaceutically acceptable salt is preferred. For example, such a salt may include an organic acid salt (e.g., a carboxylic acid salt such as an acetate, a fumarate, or a citrate; and an organic sulfonic acid salt such as a methanesulfonate), an inorganic acid salt (e.g., a chloride), a salt with an organic base (e.g., a salt with a tertiary amine, such as a trimethylamine salt or an ethanolamine salt), and a salt with an inorganic base (e.g., an ammonium salt; an alkali metal such as a sodium salt; an alkaline earth metal salt such as a calcium salt; and an aluminum salt).

The biomaterial may further contain, if necessary, an acid component (e.g., acetic acid, citric acid, tartaric acid, and malic acid), a base component (e.g., an inorganic base such as ammonia, sodium, potassium, magnesium, or calcium, and an organic base such as triethylamine, ethanolamine, or triethanolamine).

Moreover, the biomaterial or medical material may be a composite material of the polypeptide (or a composition containing the polypeptide) and an organic or inorganic base. The base usually has bioaffinity and biocompatibility in many cases.

Examples of the inorganic base may include a metal oxide such as silica, zirconia, or titania, a calcium phosphate such as hydroxyapatite, a metal such as aluminum, stainless steel, titanium or titanium alloy, and a ceramic.

The organic base may include, for example, a polysaccharide or a derivative thereof (e.g., a polysaccharide such as an alginate, a chitin, a chitosan, a hyaluronic acid, a polygalactosamine, a curdlan, a pullulan, a xanthan, or a dextran, a cellulose, a cellulose ether such as a methylcellulose, an ethylcellulose, a carboxymethylcellulose or a salt thereof, a hydroxyethylcellulose, a hydroxypropylcellulose, or a hydroxypropylmethylcellulose, and a cellulose ester such as a cellulose acetate), a protein (e.g., a gelatin, a casein, and an albumin), a polypeptide (e.g., a polylysine, a polyglutamine, and a polyglutamic acid), a vinyl alcohol-series resin (e.g., a polyvinyl alcohol-series resin, and an ethylene-vinyl alcohol copolymer), a polyvinylpyrrolidone-series resin, an acrylic resin (e.g., a (meth)acrylic acid-series resin such as a poly(meth)acrylic acid, or a (meth)acrylic acid copolymer), a halogen-containing resin (e.g., a fluorine-containing resin such as a polytetrafluoroethylene, and a vinyl chloride-series resin such as a polyvinyl chloride), a polyurethane-series resin, a silicone-series resin, a polyester-series resin, and a polyamide-series resin (e.g., a nylon 6, and a nylon 66).

The base may have a non-biodegradability or bioerodability. In the case of being used as a biomaterial, it is advantageous that the base has degradability and sorbability (or absorbability) in a living body. Such a biodegradable base may comprise a biodegradable resin. The biodegradable resin may include various resins, for example, the polysaccharide or the derivative thereof, and a polyester [e.g., a homo- or copolymer of a hydroxycarboxylic acid such as glycolic acid, lactic acid, 3-hydroxybutyric acid, 4-hydroxybutyric acid, or 3-hydroxypropionic acid (e.g., a lactic acid-glycolic acid copolyester), a copolyester of the hydroxycarboxylic acid and propionic acid and a lactone (e.g., butyrolactone, and caprolactone)].

These materials for base may be used singly or in combination, or may be a composite base using not less than two kinds of materials.

The shape or configuration of the base is not particularly limited to a specific one, and may be in the form of a particulate (e.g., a particulate having a size of about 1 to 300 μm), a one-dimensional shape (e.g., a fiber or filament form, a linear form, and a rod form), a two-dimensional shape (e.g., a film (or sheet) or a plate form), and a three-dimensional shape (e.g., a tube form) as usage. Further, the base may be either anon-porous body, or a porous body (for example, a particulate porous body, a cellulose fiber paper, a two-dimensional porous body such as a non-woven fabric or a woven fabric, and a three-dimensional porous body having a cylindrical form). If necessary, the base may be surface-treated with a finishing (or surface-treating) agent (e.g., a physiologically acceptable finishing agent).

The biomaterial of the present invention may be prepared by applying the polypeptide to at least a surface of such a base. The polypeptide may be applied to a site to be adapted to a living body in the base (a site in contact with body fluid or blood without limiting to body tissues). In a particulate or one-dimensional shaped base, the polypeptide may be applied to the entire base. In a two-dimensional shaped base, the polypeptide may be applied to at least one surface of the base. In a three-dimensional shaped base, the polypeptide may be applied to a site to be adapted to a living body (e.g., the whole area, the internal surface, and the external surface). For example, the biomaterial comprising the base having a surface coated by the polypeptide may be obtained by coating or spraying (or impregnating) the surface of the base with a solution or suspension of the polypeptide, and then drying the resulting matter. Moreover, a porous base may be impregnated with a solution or suspension of the polypeptide to give a biomaterial holding (or carrying) the polypeptide.

Further, by using a composition containing the polypeptide and the crosslinking agent, the polypeptide supported on the base may be, if necessary, crosslinked with the crosslinking agent (particularly, a physiologically acceptable crosslinking agent such as a dialdehyde (e.g., glyoxal, glutaric aldehyde, succinaldehyde, and malealdehyde), dextrandialdehyde, or aldehyde starch). The proportion of the crosslinking agent may be about 1 to 20 parts by weight, and preferably about 1 to 10 parts by weight relative to 100 parts by weight of the polypeptide, in the same manner as the above.

The amount to be applied of the polypeptide relative to the base greatly depends on the shape of the biomaterial, and is not categorically limited to a specific one. The amount may be usually about 0.1 to 500 parts by weight (e.g., about 1 to 300 parts by weight), and preferably about 5 to 200 parts by weight (e.g., about 10 to 100 parts by weight) relative to 100 parts by weight of the base.

In the case where the biomaterial or medical material of the present invention is used for medical purpose, the biomaterial or medical material is preferably sterilized or pasteurized. A sterilization or pasteurization method may include various methods, for example, pasteurization with steam such as a heated and damp steam, pasteurization with a gamma ray, pasteurization with ethylene oxide gas, sterilization with a pharmaceutical preparation, sterilization with an ultraviolet ray, and others. Among these methods, pasteurization with a gamma ray and pasteurization with ethylene oxide gas are preferred from the viewpoint of pasteurization efficiency and low (or light) adverse effects on a material to be used. Incidentally, the surface of the polypeptide applied to the base may be releasably protected by a protective film, or others.

The biomaterial (biocompatible material) or medical material of the present invention is, for example, utilized for not only a carrier or a support in tissue engineering or regeneration medical treatment (e.g., an artificial skin, a revascularization base, a lumen regeneration base, a neurotization base, a bone and cartilage regeneration base, a muscle regeneration base, a renal regeneration base, a hepar regeneration base, and an artificial eardrum material), but also a treating agent or coating agent for a prosthesis or artificial organ which brings into contact with body fluid, blood or tissue (e.g., an artificial heart, an artificial blood vessel, a bladder, an alimentary canal, a bowel membrane, a breastwork, a mediastinal space, an air tube, an artificial anus, and a joint, which are implantable in a living body). Further, the biomaterial may be effectively utilized for a coating material (or coating agent) or a liniment, an implant material (or implant agent), a hemostatic material (or hemostatic agent), an antiadhesive material (or antiadhesive agent), an adhesive material (or adhesive agent), a tubular material (e.g., a blood vessel, a urinary duct, and an enteric canal (or a bowel)), a membrane material (e.g., a peritoneum, a dura mater encephali, an artificial skin, or a prosthetic membrane material) in, for example, an affected area [e.g., a disease part or a damaged part (e.g., a damaged part such as racoma or burn injury)], or an incised area [e.g., a part cut by operation]. Incidentally, the biomaterial may be used in the form of a hydrous gel or a dry gel as usage, or may be used in the form of an absorbent pad.

The coating material (or coating agent) may also include a wound-coating material (wound-coating agent) for an external injury such as an abrasion, a cut wound or a contusion, an ambustion (or burn), an ulcer, and others, as well as an operative wound-coating material (operative wound-coating agent) for an operative wound such as a donor site or a dermabration site. The coating material (or coating agent) may be used in the form of various shapes, a strap, a film or sheet, and a liniment, depending on the shape of the wound area or the degree of the wound. The liniment may be a liquid preparation or an ointment which can be applied to an affected area or a wound area. Moreover, the coating material may be used in the form of a bandage, a gauze, an absorbent cotton, and others. In the case where the biomaterial is applied as the coating material to a wound area, the biomaterial can absorb an exudate from the wound area and hold it to inhibit emission of water, and can prevent the wound area from bacterial contamination or infection to create an environment suitable for curing.

The implant material (or implant agent) may include a wound-filling material (or wound filler) for filling a wound area, a subdermal implant (or implant agent) for subcutaneous infusion, a prosthetic filling material (or prosthetic filler), and others. The subdermal implant may be utilized as an agent for a vanity surgery in breast, nose, bucca, or chin with a view to repair a breast or body shape. The utilization of the biomaterial for such an application ensures higher biocompatibility and safety than a silicone or the like, due to the polypeptide.

The peptide is capable of forming a collagen-like tissue, and also provides a hemostatic action. Therefore, the biomaterial of the present invention is also utilized as a hemostatic material (or hemostatic agent). Incidentally, the hemostatic material may be in the form of an adhesive tape, a gauze, a bandage, and the like.

The biomaterial is also useful for an antiadhesive material (or antiadhesive agent). That is, application of the biomaterial to a removed or excised part or amputation surface of an affected area, or a defective area can effectively inhibit adhesion of the removed or excised part or the like because the polypeptide has a high biocompatibility and low adhesion on a cell adhesion site, different from a mammal-derived collagen. In particular, the biodegradable polypeptide can effectively inhibit adhesion of the removed or excised part or the like and can be absorbed in a living body, as a result, it is not necessary to remove the antiadhesive material from the applied site by an operation or other means. The antiadhesive material may be also used in various shapes such as a strap, a film or sheet, and a liniment.

Further, the biomaterial of the present invention is also useful as an adhesive material (or adhesive agent) for an incised area since the polypeptide has a high biocompatibility and adhesive ability. The adhesive material (or biological tissue adhesive agent) may be also used in various shapes such as a strap, a film or sheet, and a liniment. The adhesive material may be implanted in a living body with reducing a fractured part, or fixing or temporally tacking other tissue.

Furthermore, the biomaterial of the present invention is utilized for not only a reinforcing material of a sutural surface but also a bone-reinforcing material, a cartilage-regenerating material, and others because of a high biocompatibility.

The biomaterial or medical material of the present invention may be applied to a variety of tissues (e.g., an epidermal tissue, and a dermal tissue) of various subjects. The subject may include not only human beings, but also nonhuman animals (nonhuman animals such as monkeys, sheep, bovines, horses, dogs, cats, rabbits, rats, and mice).

(Cosmetic Preparation)

Among the bioapplicable materials, the cosmetic preparation may at least contain the polypeptide, and may be a powdery cosmetic preparation containing a powdery base material (or substrate), a solid or semisolid cosmetic preparation containing a solid or semisolid base material (an aqueous base material, a gel base material, or an oily base material), or a liquid cosmetic preparation containing a liquid base material (an aqueous or an oily base material). Moreover, the cosmetic preparation usually contains a base material (or a carrier), an effective ingredient (e.g., a moisturizer), and an additive. The polypeptide may be contained in the cosmetic preparation as at least one of these components.

The content of the polypeptide may be selected from the wide range depending on the species or dosage form of the cosmetic preparation, for example, about 0.001 to 99% by weight. In the case of using the polypeptide as a base material, the proportion of the polypeptide may be, for example, about 10 to 99% by weight, preferably about 20 to 99% by weight, and more preferably about 30 to 95% by weight relative to the whole cosmetic preparation. In the case of using the polypeptide as an effective ingredient, the proportion may be, for example, about 0.001 to 95% by weight, preferably about 0.01 to 90% by weight, and more preferably about 0.1 to 90% by weight. Moreover, in the case of using the polypeptide as an additive, the proportion may be about 0.001 to 40% by weight, preferably about 0.01 to 30% by weight, and more preferably about 0.1 to 20% by weight.

The polypeptide may be used in combination with other base material (or carrier). As the base material, various carriers described in the paragraph of the biomaterial may be used, and a base material usually employed in the cosmetic preparation, for example, a powdery base material, a solid or semisolid base material, and a liquid base material are often suitably selected depending on the dosage form.

The powdery base material may include a saccharide (e.g., a monosaccharide or a polysaccharide such as glucose, lactose, or a starch; a sugar alcohol such as sorbitol), an amino acid (e.g., serine, glycin, threonine, and alanine), a metal soap (e.g., a metal salt of a fatty acid, for example, potassium stearate, a sodium salt of a palm-oil fatty acid, magnesium myristate, and calcium stearate), a resin [for example, a thermoplastic resin such as an olefinic resin such as a polyethylene, a styrenic resin, an acrylic resin, a vinyl alcohol-series polymer, a vinyl carboxylate-series resin, a polyamide-series resin, or a polyester-series resin; and a thermosetting resin such as a phenolic resin, an amine resin (e.g., a urea resin, and a melamine resin), a thermosetting acrylic resin, an unsaturated polyester resin, an alkyd resin, an epoxy resin, or a silicone resin (e.g., a poly(methylsiloxane))], an inorganic powder component [e.g., a sericite, an extender (e.g., a natural clay mineral such as a kaolin, a talc, or a mica; a synthetic fluorphlogopite, and a hexagonal boron nitride)].

The solid or semisolid base material may include a solid or semisolid oily base material derived from plants and animals (e.g., a bees wax, a Japan tallow (or Japanese wax), a carnauba wax, a candelila wax, a cacao butter, and a beef tallow; and a lanolin), a solid or semisolid oily base material derived from a mineral (e.g., a solid paraffin, a ceresin, a microcrystalline wax; and a vaseline), and in addition, a fatty acid ester (e.g., an alkyl ester of a saturated or unsaturated fatty acid such as cetyl 2-ethylhexanoate; an alkyl ester of a saturated or unsaturated hydroxylic acid such as isostearyl malate; and an ester of a saturated fatty acid with a polyhydric alcohol such as glyceryl monostearate or ethylene glycol distearate), a higher alcohol (e.g., a saturated aliphatic alcohol such as cetyl alcohol, stearyl alcohol, or an unsaturated aliphatic alcohol such as oleyl alcohol), a higher fatty acid (e.g., stearic acid, and oleic acid), a gel base material (e.g., a viscous fluid (or substance)), and others. The viscous fluid of the gel base material may include an animal- or plant-series viscous fluid (e.g., a gum such as a queenseed gum, a tragacanth gum, or a xanthan gum; a saccharide such as a pectin, or a starch; corsican pearlwort; an alginic acid compound such as a sodium alginate, or a propylene glycol alginate; a polysaccharide such as a hyaluronic acid, a sodium chondroitin sulfate, or a chondroitin heparin; and a protein such as a soy protein, a casein, a vitronectin, a fibronectin, a keratin, an elastin, or a royal jelly), a cellulose or a derivative thereof (e.g., a cellulose; a methylcellulose, an ethylcellulose, a carboxymethylcellulose, and a hydroxyethylcellulose), a synthetic polymer (e.g., a poly(sodium acrylate), a polyvinyl alcohol, a polyvinyl methyl ether, a polyvinylpyrrolidone, a carboxyvinyl polymer, and a polyoxyalkylene glycol having a high molecular weight (e.g., a polyethylene glycol)), an inorganic viscous fluid (e.g., magnesium aluminum silicate (e.g., “BEEGUM” manufactured by Sansho CO., Ltd.)), a bentonite, an organic modified bentonite, and a swelling bentonite), and others.

The liquid base material may include an oily base material such as an oily base material (e.g., a jojoba oil, an olive oil, a cocoanut oil, a camellia oil, a macadamia nut oil, a castor oil, and squalane), a mineral-series oily base material (e.g., a liquid paraffin, a polybutene, and a silicone oil), a synthetic oily base material (e.g., a synthetic ester oil, and a synthetic polyether oil); an aqueous base material, for example, water, a water-soluble organic solvent [e.g., a lower aliphatic alcohol (e.g., ethanol, and isopropanol); an alkylene glycol (e.g., a polyoxyalkylene glycol having a low molecular weight, or a monoalkyl ester thereof, such as an ethylene glycol, diethylene glycol, propylene glycol, dipropylene glycol, 1,3-butylene glycol, a polyethylene glycol, or diethylene glycol monoethyl ester); a polyhydric alcohol such as glycerin, pentaerythritol; and a carboxylic acid such as lactic acid, or sodium pyrrolidonecarboxylate]. The bases may be used singly or in combination.

The proportion of the base may be about 10 to 99.999% by weight, preferably about 10 to 99% by weight, and more preferably about 20 to 95% by weight relative to the whole cosmetic preparation. Moreover, the proportion of the polypeptide may be about 0.001 to 500 parts by weight, preferably about 0.01 to 300 parts by weight, and more preferably about 0.1 to 100 parts by weight (e.g., about 1 to 50 parts by weight) relative to 100 parts by weight of the base.

The polypeptide may be used in combination with other effective ingredient(s). As the effective ingredient, the active ingredients exemplified in the paragraph of the biomaterial may be used, and there may be usually mentioned an astringent (e.g., a hydroxylic acid or a salt thereof, such as citric acid, lactic acid, or tartaric acid; an aluminum compound such as aluminum chloride; a zinc compound such as zinc sulfate, or sulfophenoxozinc; a proanthocyanidin; an extract of a tannin-containing plant such as hamamelis or white birch; a tansy extract, a rhubarb extract, and a horse tail extract), an emollient [e.g., an emulsified product in which an oily component (such as a triglyceride oil, a squalan, or an ester oil) is emulsified with a nonionic emulsifier (such as a monoglyceride)], a moisturizer, an emollient (e.g., salicylic acid or a derivative thereof, lactic acid, and urea), an antioxidant (e.g., tocopherol or a derivative thereof; and a polyphenol such as an anthocyanin), an ultraviolet absorbing agent or an ultraviolet-scattering inorganic pigment, a skin-whitening agent (e.g., ascorbic acid or a derivative thereof, cysteine, a placenta extract, arbutin, kojic acid, Rucinol®, ellagic acid, and a chamomile extract), an antiperspirant (e.g., an astringent such as an aluminum compound, a zinc compound, or a tannin), a dry skin inhibitor (e.g., a glycyrrhizic acid salt, and a vitamin compound), an antiinflammatory agent (e.g., allantoin, guaiazulene, glycyrrhizic acid or a salt thereof, a glycyrrhetinic acid or a salt thereof, ε-aminocaproic acid, tranexamic acid, ibuprofen, indomethacin, zinc oxide, or a derivative thereof; and a plant extract such as an arnica extract), a germicide or antibacterial agent (e.g., a quaternary ammonium salt such as benzalkonium chloride, or distearylmethylammonium chloride; a benzoic acid compound such as benzoic acid, sodium benzoate, or peroxybenzoate; a salicylic acid compound such as salicylic acid, or sodium salicylate; trichlorocarbanilide, and triclosan), an enzyme (e.g., a protease, and alipase), a vitamin compound (e.g., vitamin A, vitamin B, vitamin C, vitamin D, vitamin E, and vitamin K), an amino acid (e.g., tryptophan, and cysteine), and a cell activator (e.g., riboflavin, pyridoxine, nicotinic acid, pantothenic acid, α-tocopherol, or a derivative thereof; and a plant extract such as a strawberry geranium (Saxifraga stolonifera) extract).

Examples of the moisturizer may include an alkylene glycol (e.g., a polyalkylene glycol or a monoalkyl ester thereof, such as ethylene glycol, diethylene glycol, propylene glycol, dipropylene glycol, 1,3-butylene glycol, a polyethylene glycol, or diethylene glycolmonoethyl ester), a polyhydric alcohol such as glycerin, or pentaerythritol; lactic acid, sodium pyrrolidonecarboxylate; an amino acid (e.g., serine, glycin, threonine, and alanine); a saccharide (e.g., a sugar alcohol such as sorbitol; a polysaccharide such as a hyaluronic acid, a sodium chondroitin sulfate, or a chondroitin heparin); a protein (e.g., a vitronectin, a fibronectin, a keratin, an elastin, and a royal jelly), and others.

The ultraviolet absorbing agent may include a benzophenone-series absorbent such as oxybenzone, oxybenzonesulfonic acid, or sodium hydroxymethoxybenzophenonesulfonate; a cinnamic acid-series absorbent such as octyl methoxycinnamate, methyl diisopropylcinnamate, ethyl diisopropylcinnamate, isopropyl p-methoxycinnamate, or di-p-methoxycinnamic acid mono-2-ethylhexanoic acid glyceryl; a p-aminobenzoic acid-series absorbent such as p-aminobenzoic acid, ethyl p-aminobenzoate, octyl p-aminobenzoate, or octyl p-dimethylaminobenzoate; a salicylic acid-series absorbent such as octyl salicylate; a dibenzoylmethane-series absorbent such as 4-t-butyl-4′-methoxybenzoylmethane; urocanic acid or an ester thereof; β-isopropylfuranone; β-carotene; and others. The ultraviolet-scattering inorganic pigment may include titanium oxide (titanium dioxide), zirconium oxide, zinc oxide, iron oxide, and others. Moreover, the effective ingredient may also include, as usage, an effective ingredient of a cosmetic preparation for the hair (e.g., a hair conditioner, and a dandruff inhibitor), an effective ingredient of a cosmetic preparation for pigmented spot and fleck (e.g., a tyrosinase activation inhibitor, and a melanin-reducing agent), an effective ingredient of a cosmetic preparation for acne (e.g., a keratin softening agent such as sulfur, an antiphlogistic, a cortical hormone, and an oil secretion inhibitor), and others. The effective ingredients may be used singly or in combination.

The proportion of the effective ingredient may be about 0.001 to 90% by weight, preferably about 0.01 to 80% by weight, and more preferably about 0.1 to 60% by weight relative to the whole cosmetic preparation. Incidentally, in the case of using the polypeptide as a moisturizer, the proportion (molar ratio) of the polypeptide relative to other moisturizer (the above-mentioned moisturizer) [the polypeptide/other moisturizer] may be about 0.1/99.9 to 100/0, preferably about 1/99 to 90/10, and more preferably about 5/95 to 80/20.

In the cosmetic preparation, the polypeptide may be used in combination with other additive(s). The additive may include a surfactant (e.g., the surfactants exemplified in the paragraph of the biomaterial), an inorganic salt (e.g., sodium sulfate, sodium hydrogen carbonate, and potassium chloride), a coloring agent, a fiber (e.g., a synthetic fiber such as a nylon fiber, and a natural fiber), an abrasive (or abradant) (e.g., calcium hydrogen phosphate, calcium carbonate, and silicic anhydride), a foaming agent (e.g., sodium lauryl sulfate), a humectant or wetting agent (e.g., sorbit, and glycerin), a caking additive (a viscous fluid mentioned above, for example, a carboxymethylcellulose, a carboxymethylcellulose sodium, and a carrageenan), an opacifying agent, a perfume material (e.g., a synthetic perfume, an essential oil, and an essential oil component), a sweetening agent (e.g., saccharin sodium), a plant extract, and others.

The coloring agent (dye and pigment) may include a synthetic or natural pigment (a dye, a pigment), for example, a tar pigment, an iron oxide-series inorganic pigment, a black iron oxide lake, a white pigment such as titanium dioxide; a pearl pigment (e.g., a titanated mica system, bismuth oxychloride, and argentine); a dye such as Red No. 223, or Orange No. 201; and a natural pigment (e.g., cochineal, and carthamin).

Further, the additive may include a pH adjuster (e.g., a base such as sodium hydrogen carbonate; an acid such as sodium monohydrogen phosphate; and a borax), a chelating agent (e.g., a hydroxycarboxylic acid such as citric acid, and a phosphoric acid such as metaphosphoric acid), a metal ion sequestering agent (e.g., a polyphosphate, and ethylenediaminetetraacetate), a solidifying agent (e.g., the higher alcohols, the saturated fatty acids and the waxes which are exemplified in the paragraph of the base material), a solubilizing agent (e.g., a polyoxyethylene hydrogenated castor oil), a plasticizer [e.g., camphor, a phthalic acid ester (e.g., dibutyl phthalate), and an aliphatic polybasic acid ester such as tributyl acetylcitrate], a gelatinizing agent (e.g., an organic modified bentonite), a thickening agent (e.g., the viscous fluids exemplified in the paragraph of the above-mentioned base material), an organic solvent (e.g., an alcohol such as ethanol, or butanol), a reducing agent (e.g., thioglycolic acid or a salt thereof, and cysteine), a basic agent (e.g., an ammonia water, ammonium carbonate, and ethanolamine), an oxidizing agent (e.g., sodium bromate, hydrogen peroxide, and sodium perborate), an antiseptic or preservative (e.g., paraben, and sodium benzoate), a algefacient or refrigerant (e.g., menthol), and in addition, the additives exemplified in the paragraph of the above-mentioned biomaterial, for example, a suspending agent, a buffer, a dissolution aid, an acid component, and a base component. These additives may be used singly or in combination.

The proportion of the additive may be about 0.001 to 40% by weight, preferably about 0.01 to 30% by weight, and about 0.1 to 20% by weight relative to the whole cosmetic preparation.

Each of the base material, the effective ingredient and the additive may be used in the form of a salt. Such a salt may include the salts as mentioned in the paragraph of the biomaterial.

Incidentally, each of the base material, the effective ingredient and the additive may have an interactive property with the polypeptide as long as the properties of the polypeptide are not deteriorated. Preferably, the base, the effective ingredient and the additive do not usually have the interactive property (e.g., reactivity, and degradability).

The cosmetic preparation of the present invention may be utilized as an external composition for applying to a skin of an animal (which may be a nonhuman animal, and is usually human being). The site to be applied is not particularly limited to a specific one, and may include, for example, integuments of various sites such as head, face, neck, arm, hand, chest protection and foot, and in addition, intraoral parts, body hair such as head hair, eyelash and eyebrow, and nail.

The shape or configuration of the cosmetic preparation of the present invention is not particularly limited to a specific one, and may include, for example, a liquid preparation (e.g., a lotion, an emulsion, and a suspension), a semisolid preparation (e.g., a gel, an ointment, a plaster, and a cream), and a solid preparation (e.g., a powder, and a cake). The liquid preparation and semisolid preparation may be impregnated or applied to a base or substrate (e.g., a nonwoven fabric, a woven fabric, a paper, and a polymer film), for example, may be used as a facial mask, a mask, a wet tissue, and others.

The liquid preparation may be a solution, or a dispersion (e.g., a dispersion in which a powder is dispersed in an aqueous liquid preparation, a dispersion of a two-layer liquid preparation composed of water and a nonaqueous organic solvent, and a dispersion in which a powder is dispersed in a two-layer liquid preparation composed of water and a nonaqueous organic solvent). Moreover, the liquid preparation may be used as a spray or an aerosol. In the spray or the aerosol, the liquid preparation to be sprayed may be in the form of a fog (or mist) or a foam. Incidentally, as an aerosol propellant, a liquefied gas (e.g., a fluorocarbon, a hydrocarbon, a liquefied petroleum gas, and dimethyl ether), a compressed gas (e.g., a compressed inert gas such as nitrogen gas or carbon dioxide), and others may be used.

The type of usage of the cosmetic preparation may include a basic cosmetic preparation (e.g., a lotion, a skin lotion, a gel lotion, a milky lotion, a cream, and an essence), a makeup cosmetic preparation (e.g., a liquid or powdery foundation, a blusher, an eye shadow, and a hair dressing), a bathwater additive (e.g., a bath agent), and a cleaning agent (e.g., a facial wash, a cleansing agent, a soap, a body shampoo, a shampoo, a rinse, and a conditioner).

Moreover, the cosmetic preparation may also include, depending on the site to be applied or the application (or function), for example, a scalp and hair care cosmetic preparation (e.g., a shampoo, a hair rinse, a hair treatment, a hair essence, a hair styling agent, a perm agent, a cold wave lotion, and a hair dye), a partial cosmetic preparation [e.g., an eye makeup cosmetic preparation such as an eye liner or a mascara; a lip cosmetic preparation such as a lip balm, a lip essence, a lip rouge, a lip gloss, or a lip makeup remover; an oral cosmetic preparation (e.g., a tooth powder or a toothpaste, a mouthwash, and a mouth refrigerant); and a nail cosmetic preparation (e.g., a nail essence, a nail enamel, and an enamel remover)], a tanning or sunburn cosmetic preparation, a cosmetic preparation for pigmented spot and fleck, a cosmetic preparation for acne, and a deodorant cosmetic preparation (e.g., an antiperspirant).

The usage and dose of the cosmetic preparation of the present invention may be selected depending on the species (application) or configuration (form) of the cosmetic preparation. For example, the cosmetic preparation may be applied to a given site about once to five times a day. For example, the perm agent may be applied to a given site about once to three times a week to per several months. The enamel may be applied to a given site about once to ten times a day to a week. Moreover, for usages as the cleaning agent, the hair preparation (e.g., a rinse, a conditioner, a hair treatment, and a perm agent), and others, such a cosmetic preparation may be washed away after application.

(Food Composition)

Among the bioapplicable material, the food composition may contain at least the polypeptide, and may be any of a powdery composition containing a powdery base material, a solid or semisolid composition containing a solid or semisolid base material, a liquid composition containing a liquid base material, or a mixture thereof. Incidentally, the polypeptide may be employed as a treated matter (or substance), for example, a heat-treated matter such as a gelatin, a decomposed matter of a polypeptide or a gelatin (e.g., a collagen peptide), and others.

The food composition may usually include a base material (or carrier), an effective (active) ingredient, and an additive (e.g., a food additive, a flavor enhancer or seasoning), and others. The polypeptide may be contained in the composition as at least one component among these components.

The content of the polypeptide may be selected from a wide range of, for example, about 0.001 to 99% by weight, depending on the kind or form of the composition. In the case of using the polypeptide as a base material, the proportion of the polypeptide may be, for example, about 10 to 90% by weight, preferably about 20 to 80% by weight, and more preferably about 30 to 70% by weight relative to the whole food composition. In the case of using the polypeptide as an additive, the proportion of the polypeptide may be, for example, about 0.001 to 40% by weight, preferably about 0.01 to 30% by weight, and more preferably about 0.1 to 20% by weight. Moreover, in the case of using the polypeptide as an effective ingredient, the proportion of the polypeptide may be, for example, about 0.001 to 90% by weight, preferably about 0.01 to 80% by weight, and more preferably about 0.1 to 70% by weight.

The polypeptide may be combined with other base material(s). Among the base materials, the powdery base material may include a saccharide (e.g., a mono- or polysaccharide such as glucose, lactose, a milk sugar, a refined sugar or white sugar (saccharose), a starch, or a corn starch; a sugar alcohol such as sorbitol, xylitol, or mannitol; and a dextrin), an amino acid (e.g., serine, glycin, threonine, or alanine), a protein (e.g., a protein such as a soy protein), a polyvinylpyrrolidone, and others.

As the solid or semisolid base material, there may be exemplified that a solid or semisolid base material obtained from plants and animals (e.g., a cacao butter, a margarine, a shortening, a butter, a beef tallow, or a lard), a fatty acid ester (e.g., a saturated or unsaturated fatty acid alkyl ester such as cetyl 2-ethylhexanoate; a saturated or unsaturated hydroxylic acid alkyl ester such as isostearyl malate; an ester of a saturated fatty acid and a polyhydric alcohol, such as glyceryl monostearate, or ethylene glycol distearate), a higher alcohol (e.g., a saturated aliphatic alcohol such as cetyl alcohol, stearyl alcohol, or an unsaturated aliphatic alcohol such as oleyl alcohol), a higher fatty acid (e.g., stearic acid, and oleic acid), a gel (gelled) based material (e.g., a viscous fluid), and others. The viscous fluid may include the animal- or plant-series viscous fluids exemplified in the paragraph of the cosmetic preparation; the celluloses or derivatives thereof exemplified in the paragraph of the cosmetic preparation; a base (e.g., a chewing gum base material such as methyl acetylricinolate, or a vinyl acetate resin); and others.

The liquid base material may include an oily base material such as a soybean oil, a rapeseed oil, a cottonseed oil, a safflower oil, a peanut oil, a corn oil, a sesame oil, a grapeseed oil, an olive oil, a coconut oil or a palm nut oil; an aqueous base material such as water or ethanol; a lower carboxylic acid such as lactic acid or acetic acid.

These base materials may be used singly or in combination. The proportion of the base material may be, relative to the whole food composition, about 10 to 99.999% by weight, preferably about 10 to 99% by weight, and more preferably about 20 to 95% by weight. Moreover, the proportion of the polypeptide may be, relative to 100 parts by weight of the base material, about 0.001 to 500 parts by weight, preferably about 0.01 to 300 parts by weight, and more preferably about 0.1 to 100 parts by weight (e.g., about 1 to 50 parts by weight).

The polypeptide may be combined with other effective (active) ingredient(s). The effective ingredient may include a nutrient (nutritious component), for example, a food or feed (or fodder) material [e.g., a grain, a bean, a material derived from animals and birds (e.g., a meat, a blood, a fell (animal skin), an animal bone, or an egg), a fish (e.g., a fish food, a fish blood, a fish skin, a fish bone, or a fish roe), a milk (e.g., a cow milk), a shellfishery (including a shell), a vegetable, a forage, a fruit, a sea weed, and an insect (including a chrysalis and a spliced chrysalis)], in addition, an enzyme (e.g., lipase, collagenase, gelatinase, amylase, or lysozyme), a vitamin (e.g., vitamin A, vitamin B, vitamin C, vitamin D, vitamin E, or vitamin K), a yeast or yeast extract, a microorganism (e.g., an acidophilus), an amino acid [e.g., L-aspartic acid or a salt thereof (e.g., potassium L-aspartate, magnesium L-aspartate, or potassium magnesium L-aspartate), and aminoethylsulfonic acid (taurine)], a hormone, a protein or peptide (e.g., a silk protein, a silk peptide), a saccharide [e.g., a mono- or polysaccharide such as glucose, lactose, or fructose; a macromolecular saccharide or a salt thereof (e.g., sodium chondroitin sulfate and hyaluronate sodium); a sugar alcohol (e.g., mannitol, xylitol, and sorbitol)], and others. The effective ingredient may also include a medicinal ingredient (medicinal properties), for example, an antihistaminic agent (component) (e.g., chlorpheniramine, diphenhydramine, and salts of thereof (e.g., diphenhydramine hydrochloride), an antiallergic agent (component) (e.g., cromoglycic acid, amlexanox, or salts thereof (e.g., sodium cromoglycate)), a stomachic component, a digestive component (gastric digestant component), an antibacterial or pesticidal component (e.g., sulfoneamide such as sulfamethoxazole or a salt thereof; a quaternary ammonium or a salt thereof (e.g., benzalkonium chloride and benzethonium chloride); and ofloxacin), an acid component (e.g., acetic acid, a black vinegar, or an apple cider vinegar), a galenical component (e.g., a herbal medicinal component such as turmeric and ginseng), and others. Incidentally, the food or feed material may be employed as processed goods (artifact), for example, a crushed material (e.g., a pulverized material such as a paste or a dry powder), an expressed liquid, a pressed material, an extract, a fermented material, and others. The effective ingredient may be used singly or in combination.

The proportion of the effective ingredient may be, relative to the whole food composition, about 0.001 to 99.9% by weight, preferably about 0.01 to 95% by weight, and more preferably about 0.1 to 90% by weight.

The polypeptide may be used in combination with other additive(s). The additive may include conventional food additives or feed additives, for example, a dietary supplement (e.g., a calcium component such as calcium citrate, calcium lactate, or calcium pantothenate; ascorbic acid or a salt thereof or an ester thereof; ferric chloride; a thiamine salt; a nicotine acid compound; and a vitamin), a binder (texturizer) (e.g., pyrophosphate), a thickening agent (e.g., sodium alginate, a propylene glycol alginate, a methylcellulose, a sodium polyacrylate, or a casein), a fermentation regulant (e.g., potassium nitrate, and sodium nitrate), an alkaline chemical (e.g., an alkaline chemical for producing Chinese noodles, such as a kansui, a carbonate, or a phosphate), a sterilizer (e.g., hydrogen peroxide, hypochlorous acid, or sodium hypochlorite; a bleaching powder), an antioxidant (e.g., erythorbic acid, ascorbic acid, guaiac, dibutylhydroxytoluene, α-tocopherol, and sulfite salt), a sweetener (e.g., a starch sugar such as sucrose (cane sugar), D-xylose, or D-sorbit, a beet sugar, an oligosaccharide, a honey, or a starch syrup; glycylrrhizine; and stevioside), an acidifier or acid component (e.g., acetic acid, citric acid, tartaric acid, malic acid, gluconic acid, succinic acid, or glucono-δ-lactone), a flavor enhancer (seasoning) (e.g., sodium L-aspartate, DL-alanine, glutamic acid, monosodium succinate; a salt (sodium chloride), a soy sauce, a fermented bean paste (miso), an alcohol (e.g., a liquor (sake), and a wine, a sweet sake (mirin)), and a flavor enhancer made from fermentation with a salt), an aromatizing agent (e.g., an aroma chemical such as various esters (e.g., ethyl acetoacetate), citral, citronellal, lemon, lime, orange, or strawberry; a refrigerant agent such as peppermint or menthol; or a spice and condiment), a coloring matter (e.g., an edible dye such as β-carotin), an extract agent, a texturizing agent (e.g., D-mannitol), a color-fixing agent (e.g., sodium nitrite, sodium nitrate, and ferrous sulfate), a film-forming agent (e.g., an oxyethylene higher aliphatic alcohol, sodium oleate, and a vinyl acetate resin), a bleaching agent (e.g., sodium hydrogen sulfite), an conditioning agent (e.g., L-cysteine monohydrochloride, and calcium stearoyl lactylate), a quality improving agent (e.g., propylene glycol), an emulsifier or a suspending agent [e.g., a surfactant such as an anionic surfactant, a cationic surfactant, a nonionic surfactant, or an amphoteric surfactant, for example, a glycerin fatty acid ester, a sucrose fatty acid ester, a sorbitan fatty acid ester, a polyoxyethylene sorbitan fatty acid ester, a propylene glycol fatty acid ester; a polysaccharide such as sodium chondroitin sulfate; a water-soluble polymer, for example, a polyvinylpyrrolidone, a water-soluble cellulose ether (e.g., a methylcellulose, a hydroxyethylcellulose, a hydroxypropylcellulose, and a sodium carboxymethylcellulose); and a soy phospholipid], an enzyme, a solvent (e.g., glycerin), a preservative (e.g., benzoic acid, sorbic acid, or salts thereof), an insecticide (e.g., piperonyl butoxide), a baking powder (e.g., an ammonium alum, an alum, or ammonium chloride), a release agent (e.g., a liquid paraffin), and the like.

Further, the additive may also include a buffer (e.g., buffer solutions of phosphates or borates), a dissolution aid (e.g., a polyethylene glycol, ethanol, sodium carbonate and sodium citrate), an antiseptic agent (e.g., a p-oxybenzoic acid ester, dehydroacetic acid and sorbic acid or salts thereof), a metal ion sequestering agent (e.g., phytic acid), a basic component (an inorganic base such as ammonium hydroxide, sodium hydroxide, potassium hydroxide, magnesium hydroxide, or calcium hydroxide; an organic base such as triethylamine, ethanolamine, or triethanolamine), and the like. The additive may be used singly or in combination.

The proportion of the additive may be, relative to the whole food composition, about 0.001 to 40% by weight, preferably about 0.01 to 30% by weight, and more preferably about 0.1 to 20% by weight. In the case of using the food composition as a food product, the food additives among the additives may be employed in the designated proportion complying with Food Sanitation Law and the like. Moreover, the proportion of additives which do not have a standard to use (e.g., a natural food additive) may be suitably selected depending on the kinds of food additives or food products.

The above-mentioned base materials, effective ingredients and additives may be used in the form of a salt. Such a salt is preferred to be a physiologically or pharmaceutically acceptable salt, for example, an organic acid salt (e.g., a carboxylic acid salt such as an acetate, a fumarate, or a citrate; and an organic sulfonate such as methanesulfonate), an inorganic salt (e.g., hydrochloride), a salt with an organic base (e.g., a salt with a tertiary amine such as trimethylamine or ethanolamine), a salt with an inorganic base (e.g., an ammonium salt; an alkali metal salt such as a sodium salt; an alkaline earth metal salt such as a calcium salt; and an aluminum salt), and others.

The form of the food composition of the present invention is not particularly limited to a specific one, and may include, for example, a liquid composition, a semisolid composition (e.g., a gel, a cream, a slurry, and a paste), a solid composition (e.g., a powder, a granule, a flake, a cake, a preparation, a gumi, a nougat, a chewable tablet, and a film), and the like. The composition may be in the form of a capsule composition in which contents are encapsulated in a capsule. Moreover, the solid composition may be subjected to a coating treatment such as a sugar coating or an enteric coating.

The food composition of the present invention may be subjected to, if necessary, a conventional preservative or package treatment such as a freezing treatment (including a freeze drying), a retort treatment, or a canning treatment.

The food composition of the present invention may be a functional food such as a health food (e.g., a healthy drink such as a nutrition supplement drink), a health supplement (e.g., a nutriceutical food such as various supplements, a food aiming at improvement in eating quality), a food for specified uses [e.g., an invalid food (patient food), and a senior food (food for senior)], a food with health claims (e.g., a nutriceutical functional food, and a specified health food), and others.

Moreover, the food composition of the present invention may have the same or resembling properties with the naturally occurring collagen, for example, effects such as a furnishing of nutrition, a moisturizing action or an antiaging effect (improvement in moisturizing action inside of mouth or throat, a protection of gastrointestinal mucosa and endosporium, accerlation of producing collagen, accerlation of increasing osteoblast or fibroblast, increase of metabolism, retention of water in corneum of skin, improvement in skin fitness, prevention of wrinkle or improvement thereof, or activation of skin), strengthening of bone (including prevention of osteoporosis or improvement thereof, and improvement in bone density), and others.

(Pharmaceutical Preparation Composition)

Among the bioapplicable material, the pharmaceutical preparation composition may contain at least the polypeptide, and may be any of a solid pharmaceutical preparation, a semisolid pharmaceutical preparation, or a liquid pharmaceutical preparation. The pharmaceutical preparation of the present invention composition may be either a pharmaceutical preparation or a quasi drug, and may be utilized as preparations of variety of dosage forms. Examples of the solid pharmaceutical preparation may include a powder, a granule, a tablet, a lozenge (trochiscus), a gumi, a pill, and a capsule. Examples of the semisolid pharmaceutical preparation may include an ointment (including a cream, or an eye ointment), a cataplasm, a plaster and pressure sensitive adhesive, and a suppository. Examples of the liquid pharmaceutical preparation may include an aerosol, a suspension, an emulsion, an injectable solution, an ophthalmic solution, a lotion, and a liniment. The proportion of the polypeptide relative to the whole pharmaceutical preparation may be about 0.001 to 99% by weight, preferably about 0.01 to 95% by weight, and more preferably about 0.1 to 90% by weight.

The pharmaceutical preparation composition may usually contain an active ingredient (a pharmacologically active or physiologically active ingredient, or an effective ingredient), a carrier (including a carrier component or a base material), and an additive (or an additive component), and the polypeptide may be contained as at least one component (ingredient) among these ingredients. The proportion of each component is not particularly limited to a specific one, for example, the proportion (parts by weight) of the carrier relative to the active ingredient may be, for example, the former/the latter=about 0.01/99.99 to 95/5, preferably about 0.05/99.95 to 95/5, and more preferably about 0.1/99.9 to 90/10. Moreover, the proportion (parts by weight) of the additive relative to the active ingredient may be, for example, the former/the latter=about 0.01/99.99 to 100/0, preferably about 0.05/99.95 to 100/0, and more preferably about 0.1/99.9 to 100/0.

For example, the polypeptide is useful for an active ingredient having an action (behavior) such as moisture retention, itching-inhibitory action, improvement in cracked skin, or improvement in bone density. In particular, the polypeptide having a biodegradability with a collagenase realizes improvement in the above actions due to high absorbability in the living body.

The polypeptide as an active ingredient may be used in combination with other active ingredient(s) (or effective ingredient, or physiologically active ingredient). As the active ingredient, the active ingredients exemplified in the paragraph of the biomaterial may be used. The active ingredient may usually include, for example, a pharmacological agent affecting an autonomic nerve system (e.g., a parasympathomimetic drug, a parasympatholytic drug, a sympathetic agent, a sympatholytic agent, a ganglionic stimulating agent, or a ganglionic blocker), an autacoid and an antagonist thereof (e.g., an antihistamic, an antiserotonin agent, a kinin, or a prostaglandin), an antiinflammatory agent, an antiallergic agent, a pharmacological agent affecting a central nerve system (e.g., a hypnotic, an antiepileptic drug, an antineuropathic drug, an antianxiety agent, an antidepressant drug, or an antidepressant drug), a pharmacological agent affecting a cardiovascular system (e.g., a cardiac, an antiarrhythmic drug, an antiarteriosclerotic drug, or an antihypertensive drug), a pharmacological agent affecting a respiratory system (e.g., a sedative, an antitussive, an expectorant, or an antiasthma drug), a pharmacological agent affecting a digestive system (e.g., a stomachic, a digestant, an antacid, or an antiulcer drug), a pharmacological agent affecting a blood and a hematopoietic organ (e.g., a hemostatic, an anemia curing medicine, or an anticoagulant), a pharmacological agent affecting a skin and a mucosa (e.g., an astringent, an emollient, or a decubitus and skin curative), an antiinfective (e.g., an antimicrobial, an anthelmintic, an antiviral agent, or a fungistat), a diuretic, an ulcer curative, an antineoplastic agents, an antibiotic, a hormone drug, a vitamin, an amino acid, and a herbal medicine in many cases. These active ingredients may be used singly or in combination.

The proportion (parts by weight) of the polypeptide as an active ingredient relative to other active ingredient(s) may be, for example, the former/the latter=about 1/99 to 99/1, preferably about 5/95 to 95/5, and more preferably about 10/90 to 90/10.

The polypeptide is useful for various carriers (or base materials), for example, a solid base material (including a matrix for a sustained release product) such as a binder, an excipient, a diluent, a filler; a semisolid base material such as an emulsion base material, a suspensive base material, or a gel (or gelled) base material; and a film-forming base material (film-formable base material) such as a coating base material (e.g., a base material for forming a coat), an encapsulating base material (e.g., a base material for forming a hard capsule, a soft capsule, or a microcapsule).

The polypeptide may be used, depending on dosage form, in combination with various carriers or base materials which are physiologically acceptable. As the carrier of a solid pharmaceutical preparation, there may be exemplified a binder (e.g., the binders exemplified in the paragraph of the biomaterial, and a gelatin); an excipient (e.g., the excipients exemplified in the paragraph of the biomaterial, in addition, a sugar alcohol such as D-sorbitol, D-mannitol, or xylitol, a saccharide such as glucose or fructose, a carmellose sodium, calcium hydrogen phosphate, a starch, a dextrin, a β-cyclodextrin, titanium oxide, magnesium aluminometasilicate, a talc, and a kaolin); a disintegrator (the disintegrators exemplified in the paragraph of the biomaterial, in addition, a low-substituted hydroxypropylcellulose, and carboxymethylcellulose calcium), and the like. The solid pharmaceutical preparation may also contain a lubricant (e.g., magnesium stearate).

Moreover, the solid pharmaceutical preparation may be a coated pharmaceutical preparation which has a coating with a coating base material (e.g., a sugar-coated tablet (pill) or a coated tablet (pill)), or a capsule agent in which a solid material and that of the liquid material is encapsulated in a capsule (soft capsule or hard capsule). As the carrier of the semisolid or liquid pharmaceutical preparation, the carriers exemplified in the paragraph of the biomaterial may be used.

These carriers may be used singly or in combination. In the case of using the polypeptide as a carrier, the proportion (parts by weight) of the polypeptide relative to other carriers may be, for example, the former/the latter=about 1/99 to 99/1, preferably about 5/95 to 95/5, and more preferably about 10/90 to 90/10.

Moreover, the polypeptide may be used as an additive such as an emulsifier, a suspending agent, a stabilizer, a consistency (viscosity)-adjusting agent, a gelatinizer, and others. The polypeptide as the additive may be used in combination with other additive(s). Such an other additive may include, for example, the additives exemplified in the paragraph of the biomaterial, in addition, a pH adjuster (a base such as sodium hydrogen carbonate; an acid such as sodium hydrogenphosphate; and a borax), an organic solvent (e.g., an alcohol such as ethanol or butanol), a thickener (e.g., sodium alginate, an ester of a propylene glycol and alginic acid, a carboxymethylcellulose calcium, a carboxymethylcellulose sodium, a methylcellulose, and a sodium polyacrylate), a colorant (e.g., an edible dye and a colcothar), an isotonizing agent (e.g., sodium chloride, glucose), a pain-relieving agent (e.g., procaine hydrochloride, carbocaine hydrochloride, benzyl alcohol, chlorobutanol, and glucose), an antifoaming agent, a disintegrant auxiliary, an adsorbent, and others.

Further, if necessary, the pharmaceutical preparation composition may contain the acid and/or base components exemplified in the paragraph of the biomaterial, a bubbling (foaming) agent (e.g., sodium lauryl sulfate), a wetting agent (e.g., a sorbit, glycerin), a sweeting agent or flavoring substance (e.g., saccharine sodium, a tea extract), a fragrant material or refrigerant agent (e.g., lemon, lime, orange, menthol, or strawberry), and others.

These additives may be used singly or in combination. The proportion (weight ratio) of the polypeptide as the additive relative to other additive(s) may be, for example, the former/the latter=about 1/99 to 99/1, preferably about 5/95 to 95/5, and more preferably about 10/90 to 90/10.

Incidentally, the active ingredients, carriers and additives may be used in the form of a salt. Such a salt may include the salts exemplified in the paragraph of the biomaterial.

Incidentally, the polypeptide may be used as a film-forming base material (film-formable base material) such as a coating base material (e.g., a base material for forming a coat), an encapsulating base material (e.g., a base material for forming a hard capsule, a soft capsule, and a microcapsule) due to the high coating ability.

The above preparation may be produced in a conventional method. The solid pharmaceutical preparation may be prepared, for example, by mixing an active ingredient, various carriers (e.g., a binder, an excipient, and a disintegrant) and/or an additive, and granulating or compacting the mixture. The semisolid pharmaceutical preparation may be prepared by mixing an active ingredient, and a carrier such as an oily substance, a water-soluble polymer, or a water (aqueous) gel. The liquid pharmaceutical preparation may be obtained by dissolving, suspending, or emulsifying an active ingredient to a carrier, and the preparation may be sterilized in many cases.

Moreover, the polypeptide is useful for imparting sustained releasability to the preparation. In particular, the polypeptide having a collagenase-degradable property is useful for imparting sustained releasability to the pharmaceutical preparation composition. For example, pharmaceutical preparation compositions can be conferred sustained releasability by using the polypeptide as a carrier to form a matrix in which an active ingredient is dispersed, or to form a coating film, a soft or hard capsule, or a microcapsule coating. Moreover, in order to confer excellent sustained releasability, the polypeptide may be crosslinked by the crosslinking agents exemplified in the paragraph of the biomaterial according to need. The proportion of the crosslinking agent relative to the polypeptide can be selected from the similar range to that of the biomaterial. Incidentally, the matrix or coating may be formed by a conventional method (e.g., mixing, granulating, a coacervation method, or a spray-dry granulating method).

The pharmaceutical preparation composition of the present invention can be administered orally or parenterally (non-orally) depending on the dosage forms, for example, transdermally, transrectally, transvaginally or by injection. Moreover, the pharmaceutical preparation composition of the present invention may be applied to various subjects (e.g., the subjects exemplified in the paragraph of the biomaterial).

[Film-Forming Material (or Film-Forming Composition)]

The film-forming material or composition may be either an aqueous material (or composition), or an organic solvent (or oily) material (or composition). Moreover, the above-mentioned composition may be a solution-type composition or a suspension-type composition. The suspension-type composition may contain the polypeptide in the form of a particle, and the mean particle size of the particulate polypeptide may be, for example, about 1 to 300 μm, preferably about 2 to 100 μm, and more preferably about 3 to 50 μm, or may be not more than 1 mm (e.g., about 1 to 10 mm) depending on usages.

The film-forming composition of the present invention may contain at least the polypeptide, and the composition usually contains a base material (or carrier) and/or an additive. In such a composition, the polypeptide may be used as a base material and/or an additive. In the case of using the polypeptide as the additive, the polypeptide may be used for imparting a property (e.g., texture, appearance, touch, or function) of a natural material (e.g., human skin or leather skin). Further, even in the case of using the polypeptide as the base material, the polypeptide can also have (or combine) a function to impart a property of a natural material.

Further, the film-forming composition may contain, as the base material component, a resin component, the above-mentioned additive, a solvent, and others. As the resin component, various polymers may be employed, for example, there may be utilized a natural polymer (e.g., a polysaccharide such as a starch), a thermoplastic resin, a thermosetting resin, and others. The thermoplastic resin may include a cellulose derivative (e.g., a cellulose ester such as a cellulose acetate, or a cellulose acetate butyrate, a cellulose ether such as a methylcellulose, a ethylcellulose, a carboxymethylcellulose, a hydroxyethylcellulose, or a hydroxypropylcellulose), an olefinic resin (e.g., a chlorinated polypropylene), an acrylic resin [e.g., a homo- or copolymer of an acrylic monomer, for example, (meth)acrylic acid, a (meth)acrylic acid alkyl ester such as methyl(meth)acrylate or ethyl (meth)acrylate, hydroxyalkyl(meth)acrylate (e.g., a hydroxyC₂₋₆alkyl(meth)acrylate such as 2-hydroxyethyl (meth)acrylate or 2-hydroxypropyl(meth)acrylate), and a copolymer of the acrylic monomer with an aromatic vinyl monomer (e.g., styrene)], a vinyl-series resin [e.g., a vinyl chloride resin, a vinyl acetate-series resin (e.g., a vinyl chloride-vinyl acetate copolymer, a polyvinyl acetate, a polyvinyl alcohol, and an ethylene-vinyl alcohol copolymer)], a polyester-series resin (e.g., an aromatic homopolyester-series resin such as a polyethylene terephthalate or an aromatic copolyester-series resin, an aliphatic polyester resin such as a biodegradable polyester [e.g., a homo- or copolymer of an hydroxycarboxylic acid such as lactic acid or glycol acid, and a copolymer of the above-mentioned hydroxycarboxylic acid with a lactone), an alkyd resin], a polyamide-series resin (e.g., an aliphatic polyamide such as a polyamide 6, a polyamide 66, a polyamide 610, a polyamide 11, or a polyamide 12), a polycarbonate-series resin, and the like. The thermosetting resin may include a urethane-series resin (e.g., a urethane-series resin obtained from a diisocyanate component such as a tolylene diisocyanate, diphenylmethane diisocyanate, naphthalene diisocyanate, or hexamethylene diisocyanate with a diol component such as a polyether diol (e.g., a polyethylene glycol or a polytetramethylene ether glycol) or a polyester diol), an epoxy resin (e.g., a bisphenol A-based epoxy resin, a phenol novolak-based epoxy resin, and a hydrogenated bisphenol A-based epoxy resin), a silicone resin, an amino resin (e.g., a melamine resin, a benzoguanamine resin, and a urea resin), and the like. These resin components may be used singly or in combination.

These resin components may be suitably selected and combined with the polypeptide depending on usages, for example, in the case of producing an artificial leather, a soft resin such as a polyurethane-series resin or a polyolefinic resin may be used. In the case of producing a starch agent for a fiber product, there may be used a water-soluble polymer such as a polysaccharide, a cellulose derivative, a polyvinyl alcohol, a water-soluble polyester-series resin, a polyethylene glycol, or a water-soluble acrylic resin.

The proportion of the polypeptide in base material component (or base material) may be selected depending on usages, for example, about 1 to 100% by weight, preferably about 5 to 100% by weight, and more preferably about 10 to 100% by weight. Moreover, the proportion (weight ratio) of the polypeptide relative to the base material component (or base material) (including a resin component) may be, for example, the former/the latter=about 0.1/99.9 to 100/0, preferably about 1/99 to 100/0 (e.g., about 1/99 to 99/1), and more preferably about 5/95 to 100/0 (e.g., about 5/95 to 95/5).

The additive may include a crosslinking agent for the polypeptide (e.g., a dialdehyde such as glyoxal, glutaraldehyde or succinaldehyde, a dextran dialdehyde, or an aldehyde starch), an adhesion improver for improving adhesiveness of a polypeptide (e.g., a polyhydric alcohol such as glycerin, ethylene glycol, or propylene glycol, a saccharide such as sucrose or sorbitol, a sugar alcohol, a rosin or a derivative thereof, and a terpene or a derivative thereof), a stabilizer (e.g., an antioxidant, an ultraviolet ray absorbing agent, and a heat stabilizer), a plasticizer, a thickening agent, a dispersing agent, a wetting agent, a defoaming agent, an antiseptic agent, a fluorescent brightener, a fragrant material, a hardening accelerator, a leveling agent, a lubricant, a flame retardant, an antistatic agent, and others. The crosslinking agent is useful for enhancing film-forming or adhesive properties of the film-forming composition. These additives may be suitably selected depending on the species of the film-forming composition, and may be used singly or in combination. The proportions of these additives may be selected depending on usage or purpose. For example, the proportion of the crosslinking agent or the adhesion improver may be, relative to of 100 parts by weight or the polypeptide, about 1 to 20 parts by weight, and preferably about 1 to 10 parts by weight. Incidentally, in the case of using the polypeptide as an additive, the proportion (weight ratio) of the peptide relative to the additive component(s) may be, for example, the former/the latter=about 0.1/99.9 to 100/0, preferably about 1/99 to 100/0, and more preferably about 5/95 to 100/0 (e.g., about 5/95 to 95/5).

Further, the film-forming composition may contain a coloring agent (a dye and pigment), a filler, and others.

The aqueous solvent contained in the aqueous composition may be not limited to a specific one, for example, the aqueous solvent may include water, an alcohol (e.g., ethanol, isopropanol, or hexafluoroisopropanol), a ketone (e.g., acetone), a sulfoxide (e.g., dimethyl sulfoxide), an amide (e.g., dimethylformamide, dimethylacetoamide, or N-methylpyrrolidone), and the like. These aqueous solvents may be used singly or in combination.

The organic solvent contained in the oily composition may be not limited to a specific one, for example, the organic solvent may include a halogenated hydrocarbon (e.g., methylene chloride), an ether (e.g., diethyl ether), an ester (e.g., ethyl acetate, butyl acetate), a ketone (e.g., methyl ethyl ketone), an aliphatic hydrocarbon (e.g., hexane), an alicyclic hydrocarbon (e.g., cyclohexane), an aromatic hydrocarbon (e.g., toluene, xylene), an amide (e.g., dimethylformamide), a nitrile (e.g., acetonitrile), and others. These organic solvents may be used singly or in combination.

As the substrate (or base) for applying the film-forming composition of the present invention, for example, there may be mentioned various substrates or molded articles made from natural polymers, plastics, ceramics, or metals. The configuration of the molded articles may be a particulate structure, a filiform (linear) or fibrous structure, a two-dimensional structure such as film-like or sheet-like structure, or a three-dimensional structure. Specifically, the substrate may include a fiber such as a natural fiber (e.g., a paper, a silk, a wool, a cotton, or a flax or hemp), or a synthesized fiber (e.g., a polyamide fiber, a polyester fiber, or an acrylic fiber), or a fabric thereof (e.g., a nonwoven fabric or a woven fabric) or a fiber product thereof (e.g., clothes such as a cutter shirt, a blouse, or pants; bedclothes such as a sheet), a porous substrate (e.g., a paper, a natural or synthetic wood, or a leather (natural leather) or artificial leather), an article of daily use (e.g., a domestic electric appliance or an interior material for automotive trim), and others.

The film-forming composition may include a coating or covering agent and an adhesive. Examples of the coating or covering agent may include, for example, a paint, a coating agent, an starch agent, a coating or finishing agent (surface treating agent), and others. In the case of using the composition of the present invention as a coating agent or a paint, the composition can be applied to wide range of substrates. The coating agent or the paint may be usually applied on the surface of a substrate and dried to form a coat. Incidentally, the coat may be also pre-formed with a coating agent or a paint, and may be adhered to a substrate through an adhesive. The starch agent may be mainly used for the fiber product. Application of the starch agent to the surface of the fiber product realizes keeping (adjusting) the shape of the fiber product, as well as imparting smoothness or antifouling property to the fiber product. The coating or finishing agent (surface treating agent) may be applied or sprayed to the surface of the substrate (e.g., the fiber products, the leather or artificial leather, or the article of daily use), or the above substrate may be infiltrated (immersed) in the coating or finishing agent (surface treating agent). Thereby, the above-mentioned polypeptide can be adhered (or attached) to the substrate surface, and the surface can be reformed or modified. The coating or finishing agent (surface treating agent) may usually employ an aqueous solvent in many cases. The adhesive agent may be used for adhesion of a porous substrates (e.g., a paper, a natural or synthetic wood or timber, or a fiber or fibrous material), and can adhere the substrate in high degree of adhesion.

In the bioapplicable materials (or bioapplicable compositions, for example, the biomaterials or biocompatible materials, the cosmetic preparations, the food compositions, the pharmaceutical preparation compositions) of the present invention, since the specific synthetic polypeptide which is capable of forming a collagen-like three-dimensional structure and tissue structure is used, such bioapplicable materials are free from a risk of an infection by a pathogenic organism or a risk of a transmission of a causative (pathogenic) factor, have a high safety, and a high bioaffinity and biocompatibility.

Since the bioapplicable materials or the film-forming materials of the present invention contain the specific synthetic polypeptide having collagen-like properties, the bioapplicable materials or the film-forming materials not only have excellent collagen-like properties, but also are free from a risk of an infection by a pathogenic organism or a risk of a transmission of a pathogenic factor with high safety.

The biomaterials or biocompatible materials are free from a risk of an undesired side effect, and further is degradable with a collagenase and sorbable (absorbable or resorbable) in a living body. Moreover, the cosmetic preparations are high in moisturizing property and stability, and free from generation of an offensive smell. Further, the synthetic polypeptides contribute to downward price of the cosmetic preparation.

Moreover, use of the synthetic polypeptide having a collagenase-degradability ensures to enhance sorbability of the food compositions in a living body, as well as to impart sustained releasability to the pharmaceutical preparation composition.

Furthermore, the film-forming materials (or compositions) have a high adhesiveness to a base, and excellent in biodegradability resulting in lessening environmental load.

Since the materials or compositions of the present invention contain the specific synthetic polypeptide, the materials or compositions are useful for various applications.

Since the synthetic polypeptides have high affinity and compatibility with high safety, the synthetic polypeptide is useful for a bioapplicable material (or bioapplicable composition) containing the synthetic polypeptide, for example, biomaterials (or biocompatible materials) or medical materials (e.g., coating materials or liniments, implants, hemostatic materials, antiadhesive materials, adhesive materials, tube members, and membrane materials), various cosmetic preparations (e.g., basic cosmetic preparations, makeup cosmetic preparations, bathwater additives, and cleaning or washing agents), food compositions [e.g., various foods, in addition, functional foods, for example, health foods, health supplements, functional foods, foods for specified uses, or foods with health claims; animal feeding stuffs for domestic animals (e.g., cattle, pigs, and sheep), pet animals (e.g., mammals such as dogs and cats, birds (or poultry), and reptiles), fish (e.g., cultured fish such as sea breams and eels; and aquarium fishes such as goldfishes), and experimental (or laboratory) animals (e.g., rats)], pharmaceutical preparation compositions (e.g., solid pharmaceutical preparations, semisolid pharmaceutical preparations, or liquid pharmaceutical preparations), and others. Incidentally, the cosmetic preparation may be utilized, depending the site to be applied or the function, for scalp and hair care cosmetic preparations, partial cosmetic preparations (e.g., eye makeup cosmetic preparations, lip cosmetic preparations, oral cosmetic preparations, and nail cosmetic preparations), in addition, tanning or sunburn cosmetic preparations, cosmetic preparations for pigmented spot and fleck, cosmetic preparations for acne, deodorant cosmetic preparations and others.

Moreover, the polypeptide is excellent in not only safety or stability (heat stability), but also adhesive properties to substrates (or base) comparing to the mammal-derived collagen. Accordingly, the film-forming material (or composition) containing the polypeptide is useful for, for example, coating agents or adhesive agents.

### EXAMPLES

The following examples are intended to describe this invention in further detail and should by no means be interpreted as defining the scope of the invention.

### Production Example 1

A peptide (5 mg (0.002 mmol)) represented by the formula: H-(Pro-Pro-Gly)₁₀-OH (Sequence ID: 1; manufactured by Peptide Institute, Inc.) was suspended in 2 mL of dimethyl sulfoxide, and the mixture was stirred at a room temperature. To the mixture were added 0.31 mg (0.0024 mmol) of diisopropylethylamine, 0.32 mg (0.0024 mmol) of 1-hydroxybenzotriazole, and 0.46 mg (0.0024 mmol) of 1-ethyl-3-(3-dimethylaminopropyl)-carbodiimide hydrochloride, and the resulting mixture was further stirred for 7 days at a room temperature.

The reaction solution was diluted 20-fold with water, and the diluted solution was subjected to a gel-permeation chromatography (AKTA purifier system, manufactured by Amarsham Bioscience K.K., column: Superdex 200 HR 10/30, flow rate: 0.5 mL/min., eluent: 10 mM phosphate buffer (pH 7.4) containing 150 mM NaCl), and the peak of the molecular weight of the polypeptide was confirmed in the range from 40,000 to 200,000 in the molecular weight distribution. The molecular weight was calculated with a Gel Filtration LMW Calibration Kit and a Gel Filtration HMW Calibration Kit (manufactured by Amarsham Bioscience K.K.) as a reference material.

The obtained reaction solution was diluted 5-fold with water, and the diluted solution was dialyzed against water for 3 days for removing a reagent (such as a condensing agent) and an unreacted monomer to give a polypeptide. The circular dichroism spectrum of the obtained polypeptide was measured, and positive Cotton effect was observed at a wavelength of 227 nm and negative Cotton effect at a wavelength of 199 nm. The results confirmed that the polypeptide formed a triple helical structure. The obtained polypeptide was denoted as a polypeptide (Ia).

### Production Example 2

A peptide chain represented by the formula: H-(Pro-Pro-Gly)₅-OH (Sequence ID: 2) was synthesized by a solid-phase synthesis with an automatic peptide synthesis machine. That is, with the use of 0.1 mmol of a particulate resin [HMP glycine, manufactured by Applied Biosystems (US)] which comprised a styrene-divinylbenzene copolymer [molar ratio of styrene relative to divinylbenzene: 99/1] containing 4-(N^{α}-9-(fluorenylmethoxycarbonyl)-glycine)-oxymethyl-phenoxy-methyl group in a proportion of 0.65 mmol/g (resin), the carboxyl terminal of one amino acid was sequentially linked (or bound) to the amino terminal of the other amino acid so as to give an object peptide. In this link reaction, 1 mmol of N^{α}-9-(fluorenylmethoxycarbonyl)-L-proline [Fmoc proline] and 1 mmol of N^{α}-9-(fluorenylmethoxycarbonyl)-glycine [Fmoc glycine], (each manufactured by Applied Biosystems (US)) were used as amino acids in each linking step.

The obtained peptide resin (resin binding the peptide) was suspended in 10 mL of dimethylformamide, and to the mixture were added 50 mg (0.5 mmol) of succinic anhydride and 13 mg (0.1 mmol) of diisopropylethylamine, and the resulting mixture was allowed to react for 12 hours at a room temperature. Thereafter, the resultant washed alternately with methyl alcohol and dichloromethane, and dried under a reduced pressure to give a peptide resin.

The obtained peptide resin was treated with 10 mL of trifluoroacetic acid containing 5% water for 3 hours. The resulting solution was added to diethyl ether to form a precipitate, and the precipitate was further washed with diethyl ether several times to deprotect the peptide and to eliminate the peptide from the resin. The resulting crude product was purified by a PD10 column (manufactured by Amarsham Bioscience K.K.) to give a peptide. The purified peptide obtained in the foregoing manner was subjected to a column chromatography [“AKTA explorer10XT” manufactured by Amarsham Bioscience K.K., column: “Nova-Pak C18”, manufactured by Millipore Corporation, 3.9 mmφ×150 mm, mobile phase: a mixed solvent of acetonitrile and water containing 0.05 vol. % of trifluoroacetic acid (concentration of acetonitrile was linearly increased from 5 to 50 vol. % for 30 minutes), flow rate: 1.0 mL/min.], and a single peak was shown at a retention time of 14.5 minutes. The molecular weight of the purified polypeptide was determined as 1375 based on FAB method mass spectrum (theoretical value: 1374.52).

A peptide (1.4 mg (0.001 mmol)) represented by the formula: HOOC—(CH₂)₂—CO-(Pro-Pro-Gly)₅-OH and ethylenediamine (0.06 mg (0.001 mmol)) were suspended in 0.05 mL of water, and to the mixture were added 0.32 mg (0.0024 mmol) of 1-hydroxybenzotriazole and 4.6 mg (0.024 mmol) of 1-ethyl-3-(3-dimethylaminopropyl)-carbodiimide hydrochloride, and the resulting mixture was shaken for 3 days at a room temperature.

The reaction solution was diluted 100-fold with water, and the diluted solution was subjected to a gel-permeation chromatography (AKTA purifier system, manufactured by Amarsham Bioscience K.K., column: Superdex 200 HR 10/30, flow rate: 0.5 mL/min., eluent: 10 mM phosphate buffer (pH 7.4) containing 150 mM NaCl) for measuring a molecular weight, and the peak of the molecular weight of the polypeptide was confirmed in the range from 30,000 to 200,000 in the molecular weight distribution. The molecular weight was calculated with a Gel Filtration LMW Calibration Kit and a Gel Filtration HMW Calibration Kit (manufactured by Amarsham Bioscience K.K.) as a reference material.

The obtained reaction solution was diluted 5-fold with water, and the diluted solution was dialyzed against water for 3 days for removing a reagent (such as a condensing agent) and an unreacted monomer to give a polypeptide. The circular dichroism spectrum of the obtained polypeptide was measured, and positive Cotton effect was observed at a wavelength of 228 nm and negative Cotton effect at a wavelength of 198 nm. The results confirmed that the polypeptide formed a triple helical structure. The obtained polypeptide was denoted as a polypeptide (Ib).

### Production Example 3

A peptide (5 mg (0.0016 mmol)) represented by the formula: H-(Pro-Hyp-Gly)₁₀-OH (Sequence ID: 3; manufactured by Peptide Institute, Inc.) was suspended in 2 mL of dimethyl sulfoxide, and the mixture was stirred at a room temperature. To the mixture were added 0.23 mg (0.0018 mmol) of diisopropylethylamine, 0.24 mg (0.0018 mmol) of 1-hydroxybenzotriazole, and 0.65 mg (0.0034 mmol) of 1-ethyl-3-(3-dimethylaminopropyl)-carbodiimide hydrochloride, and the resulting mixture was further stirred for 7 days at a room temperature.

The reaction solution was diluted 20-fold with water, and the diluted solution was subjected to a gel-permeation chromatography (AKTA purifier system, manufactured by Amarsham Bioscience K.K., column: Superdex 200 HR 10/30, flow rate: 0.5 mL/min., eluent: 10 mM phosphate buffer (pH 7.4) containing 150 mM NaCl), and the peak of the molecular weight of the polypeptide was confirmed in the range from 60,000 to 200,000 and over in the molecular weight distribution. The molecular weight was calculated with a Gel Filtration LMW Calibration Kit and a Gel Filtration HMW Calibration Kit (manufactured by Amarsham Bioscience K.K.) as a reference material.

The obtained reaction solution was diluted 5-fold with water, and the diluted solution was dialyzed against water for 3 days for removing a reagent (such as a condensing agent) and an unreacted monomer to give a polypeptide. The circular dichroism spectrum of the obtained polypeptide was measured, and positive Cotton effect was observed at a wavelength of 225 nm and negative Cotton effect at a wavelength of 197 nm. The results confirmed that the polypeptide formed a triple helical structure. The obtained polypeptide was denoted as a polypeptide (Ic).

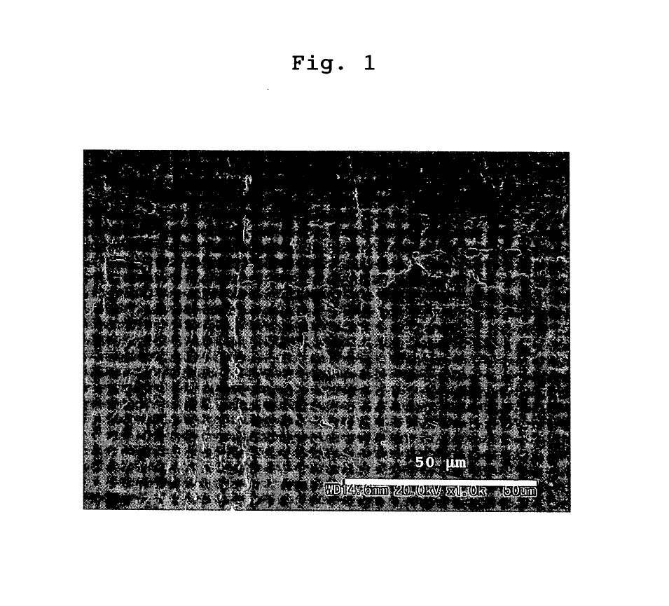

A water suspension of the obtained polypeptide (Ic) was cast on a sheet of a fluorine resin (polytetrafluoroethyene), and the cast matter was air-dried to produce a cast film. After depositing the cast film with gold, the gold-deposited cast film was observed with a scanning electron microscope. The observation revealed that the cast film had a fibrous structure as shown in FIG. 1.

### Production Example 4

A peptide (3.5 mg (0.0026 mmol)) represented by the formula: H-(Pro-Pro-Gly)₅-OH (Sequence ID: 2; manufactured by Peptide Institute, Inc.) and a peptide (0.92 mg (0.0011 mmol)) represented by the formula: H-(Val-Pro-Gly-Val-Gly)₂-OH (Sequence ID:4) which was synthesized in the same manner with the Example 2, were suspended in 1.5 mL of dimethyl sulfoxide in a give proportion (the former:the latter=70% by mol:30% by mol), and the mixture was stirred at a room temperature. To the mixture were added 0.52 mg (0.0040 mmol) of diisopropylethylamine, 0.51 mg (0.0038 mmol) of 1-hydroxybenzotriazole, and 1.45 mg (0.0076 mmol) of 1-ethyl-3-(3-dimethylaminopropyl)-carbodiimide hydrochloride, and the resulting mixture was further stirred for 7 days at a room temperature.

The reaction solution was diluted 20-fold with water, and the diluted solution was subjected to a gel-permeation chromatography (AKTA purifier system, manufactured by Amarsham Bioscience K.K., column: Superdex 200 HR 10/30, flow rate: 0.5 mL/min., eluent: 10 mM phosphate buffer (pH 7.4) containing 150 mM NaCl), and the peak of the molecular weight of the polypeptide was confirmed in the range from 80,000 to 450,000 in the molecular weight distribution. The molecular weight was calculated with a Gel Filtration LMW Calibration Kit and a Gel Filtration HMW Calibration Kit (manufactured by Amarsham Bioscience K.K.) as a reference material.

The obtained reaction solution was diluted 5-fold with water, and the diluted solution was dialyzed against water for 3 days for removing a reagent (such as a condensing agent) and an unreacted monomer to give a polypeptide. The circular dichroism spectrum of the obtained polypeptide was measured, and positive Cotton effect was observed at a wavelength of 227 nm and negative Cotton effect at a wavelength of 198 nm. The results confirmed that the polypeptide formed a triple helical structure. The obtained polypeptide was denoted as a polypeptide (Id).

After a water suspension of the obtained polypeptide (Id) was cast on a sheet of a fluorine resin (polytetrafluoroethylene), the cast matter was air-dried to produce a cast film. By immersing the cast film into 10 mM phosphate buffer (pH 7.4) containing 150 mM NaCl, a sheet-like gel substance was obtained. The sheet-like gel substance was transparent at a room temperature, and became clouded reversibly at a temperature of not lower than 40° C.

### Production Example 5

A peptide (5 mg (0.0033 mmol)) represented by the formula: H-(Pro-Hyp-Gly)₅-OH (Sequence ID: 5; manufactured by Peptide Institute, Inc.) was suspended in 2 mL of dimethyl sulfoxide, and the mixture was stirred at a room temperature. To the mixture were added 0.44 mg (0.0034 mmol) of diisopropylethylamine, 0.46 mg (0.0033 mmol) of 1-hydroxybenzotriazole, and 1.3 mg (0.0068 mmol) of 1-ethyl-3-(3-dimethylaminopropyl)-carbodiimide hydrochloride, and the resulting mixture was further stirred for 14 days at a room temperature.

The reaction solution was diluted 20-fold with water, and the diluted solution was subjected to a gel-permeation chromatography (AKTA purifier system, manufactured by Amarsham Bioscience K.K., column: Superdex 200 HR 10/30, flow rate: 0.5 mL/min., eluent: 10 mM phosphate buffer (pH 7.4) containing 150 mM NaCl), and the peak of the molecular weight of the polypeptide was confirmed in the range from 40,000 to 100,000 and over in the molecular weight distribution. The molecular weight was calculated with a Gel Filtration LMW Calibration Kit and a Gel Filtration HMW Calibration Kit (manufactured by Amarsham Bioscience K.K.) as a reference material.

The obtained reaction solution was diluted 5-fold with water, and the diluted solution was dialyzed against water for 3 days for removing a reagent (such as a condensing agent) and an unreacted monomer to give a polypeptide. The circular dichroism spectrum of the obtained polypeptide was measured, and positive Cotton effect was observed at a wavelength of 224 nm and negative Cotton effect at a wavelength of 199 nm. The results confirmed that the polypeptide formed a triple helical structure. The obtained polypeptide was denoted as a polypeptide (Ie).

### Production Example 6

A peptide (5 mg (0.0016 mmol)) represented by the formula: H-(Pro-Hyp-Gly)₁₀-OH (Sequence ID: 3; manufactured by Peptide Institute, Inc.) was dissolved in 0.5 mL of 10 mM phosphate buffer (containing 8.1 mM of Na₂HPO₄.12H₂O, 1.5 mM of KH₂PO₄, and 2.7 mM of KCl; pH 7.4), and the mixture was stirred at 20° C. To the mixture were added 0.24 mg (0.0018 mmol) of 1-hydroxybenzotriazole, and 31 mg (0.16 mmol) of 1-ethyl-3-(3-dimethylaminopropyl)-carbodiimide hydrochloride, and the resulting mixture was further stirred for 24 hours at 20° C.

The reaction solution was diluted 60-fold with water, and the diluted solution was subjected to a gel-permeation chromatography (AKTA purifier system, manufactured by Amarsham Bioscience K.K., column: Superose 6 HR 10/30, flow rate: 0.5 mL/min., eluent: 10 mM phosphate buffer (pH 7.4) containing 150 mM NaCl), and the peak of the molecular weight of the polypeptide corresponding to the average molecular weight of 400,000 was confirmed. The molecular weight was calculated with a Gel Filtration LMW Calibration Kit and a Gel Filtration HMW Calibration Kit (manufactured by Amarsham Bioscience K.K.) as a reference material.

The obtained reaction solution was diluted 5-fold with water, and the diluted solution was dialyzed against water for 3 days for removing a reagent (such as a condensing agent) and an unreacted monomer to give a polypeptide. The circular dichroism spectrum of the obtained polypeptide was measured, and positive Cotton effect was observed at a wavelength of 225 nm and negative Cotton effect at a wavelength of 197 nm. The results confirmed that the polypeptide formed a triple helical structure. The obtained polypeptide was denoted as a polypeptide (If).

### Production Example 7

A peptide (1 g) represented by the formula: H-(Pro-Hyp-Gly)₁-OH (manufactured by Peptide Institute, Inc.) was dissolved in 20 mL of 10 mM phosphate buffer (pH 7.4). To the mixture were added 473 mg of 1-hydroxybenzotriazole, and 3.35 g of 1-ethyl-3-(3-dimethylaminopropyl)-carbodiimide hydrochloride, and the resulting mixture was stirred for 2 hours at 4° C., followed by stirred for 46 hours at 20° C. The reaction solution was dialyzed against Milli Q water (ultrapure water) for 48 hours.

The obtained dialyzed solution was diluted 50-fold with water, and the diluted solution was subjected to a gel-permeation chromatography (AKTA purifier system, manufactured by Amarsham Bioscience K.K., column: Superdex 200 HR 10/30, flowrate: 0.5 mL/min., eluent: 10 mM phosphate buffer (pH 7.4) containing 150 mM NaCl), and the peak of the molecular weight of the polypeptide was confirmed in the range from 100,000 to 600,000 in the molecular weight distribution.

Moreover, the obtained dialyzed solution was diluted 100-fold with water, and the circular dichroism spectrum of the obtained polypeptide was measured. As a result, positive Cotton effect was observed at a wavelength of 225 nm and negative Cotton effect at a wavelength of 198 nm. The results confirmed that the polypeptide formed a triple helical structure.

The concentration of the obtained peptide having a triple helical structure was measured with a working curve derived from absorbance of a peptide represented by the formula: H-(Pro-Hyp-Gly)₁₀-OH (Sequence ID: 3; manufactured by Peptide Institute, Inc.) at 215 nm. The obtained polypeptide had a concentration of about 20 mg/mL. The obtained polypeptide was denoted as a polypeptide (Ih).

### Production Example 8

A peptide chain represented by the formula: H-(Pro-Hyp-Gly)₄-Pro-Gln-Gly-Ile-Ala-Gly-(Pro-Hyp-Gly)₄-OH (Sequence ID: 6) was synthesized by a solid-phase synthesis with an automatic peptide synthesis machine. That is, with the use of 0.1 mmol of a particulate resin [HMP glycine, manufactured by Applied Biosystems (US)] which comprised a styrene-divinylbenzene copolymer [molar ratio of styrene relative to divinylbenzene: 99/1] containing 4-(N^{α}-9-(fluorenylmethoxycarbonyl)-glycine)-oxymethyl-phenoxy-methyl group in a proportion of 0.65 mmol/g (resin), the carboxyl terminal of one amino acid was sequentially linked (or bound) to the amino terminal of the other amino acid so as to give an object peptide. In this link reaction, 1 mmol of N^{α}-9-(fluorenylmethoxycarbonyl)-L-proline[Fmoc proline], 1 mmol of N^{α}-9-(fluorenylmethoxycarbonyl)-glycine [Fmoc glycine], 1 mmol of N^{α}-9-(fluorenylmethoxycarbonyl)-N^{γ}-trityl-L-glutamine [Fmoc glutamine], 1 mmol of N^{α}-9-(fluorenylmethoxycarbonyl)-L-isoleucine [Fmoc isoleucine], and 1 mmol of N^{α}-9-(fluorenylmethoxycarbonyl)-L-alanine [Fmoc alanine] (each manufactured by Applied Biosystems (US)), and 1 mmol of N^{α}-9-(fluorenylmethoxycarbonyl)-O-t-butyl-L-hydroxyproline [Fmoc hydroxyproline] (manufactured by Bachem AG) were used as amino acids in each linking step.

The peptide resin obtained by the above-mentioned manner was treated with 10 mL of trifluoroacetic acid containing 5% water for 3 hours. The resulting solution was added to diethyl ether to form a precipitate, and the precipitate was further washed with diethyl ether several times to deprotect the peptide and to eliminate the peptide from the resin. The resulting crude product was purified by a PD10 column (manufactured by Amarsham Bioscience K.K.) to give a peptide. The purified peptide obtained in the foregoing manner was subjected to a column chromatography [“AKTA explorer10XT” manufactured by Amarsham Bioscience K.K., column: “Nova-Pak C18”, manufactured by Millipore: Corporation, 3.9 mmφ×150 mm, mobile phase: a mixed solvent of acetonitrile and water containing 0.05 vol. % of trifluoroacetic acid (concentration of acetonitrile was linearly increased from 5 to 50 vol. % for 30 minutes), flow rate: 1.0 mL/min.], and a single peak was shown at a retention time of 12.4 minutes. The molecular weight of the purified peptide was determined as 2681.3 based on FAB method mass spectrum (theoretical value: 2679.9).

The peptide (2.5 mg (0.0009 mmol)) represented by the formula: H-(Pro-Hyp-Gly)₄-Pro-Gln-Gly-Ile-Ala-Gly-(Pro-Hyp-Gly)₄-OH was suspended in 1 mL of dimethyl sulfoxide with stirring at a room temperature. To the mixture were added 0.12 mg (0.0009 mmol) of diisopropylethylamine, 0.12 mg (0.0009 mmol) of 1-hydroxybenzotriazole and 0.34 mg (0.0018 mmol) of 1-ethyl-3-(3-dimethylaminopropyl)-carbodiimide hydrochloride, and the resulting mixture was further stirred for 2 days at 20° C. The obtained solution was diluted 3-fold with water, and the diluted solution was dialyzed against water for 3 days for removing a reagent (such as a condensing agent) and an unreacted monomer to give a polypeptide (IIa). The proportion of the peptide unit (4) relative to the peptide unit (5) [(4)/(5)] was 8/1 (88.9/11.1) (molar ratio).

The obtained polypeptide (IIa) was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was confirmed in the range from 70,000 to 1,000,000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide (IIa) was measured, and positive Cotton effect was observed at a wavelength of 223 nm and negative Cotton effect at a wavelength of 198 nm. The results confirmed that the polypeptide formed a triple helical structure.

### Production Example 9

A peptide (1.2 mg (0.00045 mmol)) represented by the formula: H-(Pro-Hyp-Gly)₁₀-OH (Sequence ID: 3; manufactured by Peptide Institute, Inc.) and a peptide (1.2 mg (0.00045 mmol)) represented by the formula: H-(Pro-Hyp-Gly)₄-Pro-Gln-Gly-Ile-Ala-Gly-(Pro-Hyp-Gly)₄-OH (Sequence ID: 6) which was obtained in the Production Example 8 were dissolved in 0.25 mL of 10 mM phosphate buffer solution (pH 7.4). To the peptide solution were added 0.12 mg (0.0009 mmol) of 1-hydroxybenzotriazole and 15.7 mg (0.082 mmol) of 1-ethyl-3-(3-dimethylaminopropyl)-carbodiimide hydrochloride, and the resulting mixture was further stirred for 2 days at 20° C. The reaction solution was diluted 10-fold with water, and the diluted solution was dialyzed against water for 3 days for removing a reagent (such as a condensing agent) and an unreacted monomer to give a polypeptide (IIb). The proportion of the peptide unit (4) relative to the peptide unit (5) [(4)/(5)] was 18/1 (94.7/5.3) (molar ratio).

The resulting polypeptide (IIb) was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was confirmed in the range from 140,000 to 1,000,000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide (IIb) was measured, and positive Cotton effect was observed at a wavelength of 224 nm and negative Cotton effect at a wavelength of 196 nm. The results confirmed that the polypeptide formed a triple helical structure.

### Production Example 10

A peptide (2.2 mg (0.00081 mmol)) represented by the formula: H-(Pro-Hyp-Gly)₁₀-OH (Sequence ID: 3; manufactured by Peptide Institute, Inc.) and a peptide (0.24 mg (0.00009 mmol)) represented by the formula: H-(Pro-Hyp-Gly)₄-Pro-Gln-Gly-Ile-Ala-Gly-(Pro-Hyp-Gly)₄-OH (Sequence ID: 6) which was obtained in the Production Example 8 were dissolved in 0.25 mL of 10 mM phosphate buffer solution (pH 7.4). To the peptide solution were added 0.12 mg (0.0009 mmol) of 1-hydroxybenzotriazole and 15.7 mg (0.082 mmol) of 1-ethyl-3-(3-dimethylaminopropyl)-carbodiimide hydrochloride, and the resulting mixture was further stirred for 2 days at 20° C. The reaction solution was diluted 10-fold with water, and the diluted solution was dialyzed against water for 3 days for removing a reagent (such as a condensing agent) and an unreacted monomer to give a polypeptide (IIc). The proportion of the peptide unit (4) relative to the peptide unit (5) [(4)/(5)] was 98/1 (=about 99/1) (molar ratio).

The resulting polypeptide (IIc) was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing NaCl in a concentration of 150 mM), and the peak of the molecular weight of the polypeptide was confirmed in the range from 140,000 to 400,000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide (IIc) was measured, and positive Cotton effect was observed at a wavelength of 224 nm and negative Cotton effect at a wavelength of 197 nm. The results confirmed that the polypeptide formed a triple helical structure.

### Test Example 1

Each of the polypeptides (0.025 mg) obtained in the Production Examples 8 to 10 was dissolved in 0.05 mL of 50 mM Tris/HCl buffer (pH=7.5) containing 50 mM NaCl and 10 mM CaCl₂. Further, to each of the solutions was added 200 ng of collagenase (MMP-1, human rheumatoid synovial fibroblast) which was dissolved in 0.05 mL of 50 mM Tris/HCl buffer (pH=7.5) containing NaCl (50 mM) and CaCl₂ (10 mM). The resulting mixture was allowed to stand at 37° C. for 24 hours. Then, 0.1 M HCl aqueous solution (0.01 mL) was added to the mixture to stop the enzyme reaction. The mixture was diluted with 10 mM phosphate buffer (pH 7.4) containing NaCl (150 mM), and subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing NaCl (150 mM)) to measure a change in the molecular weight distribution.

As a result, the molecular weight peak of the polypeptide of the Production Example 8 was reduced to about 540,000 by adding a collagenase, compared with about 1,000,000 in the case of not adding a collagenase. In the same manner, by adding a collagenase, the molecular weight peak of the polypeptide of the Production Example 9 was reduced from about 800,000 to about 400,000, and the molecular weight peak of the polypeptide of the Production Example 10 was reduced from about 700,000 to about 300,000.

### Production Example 11

A peptide chain represented by the formula: H-(Pro-Hyp-Gly)₄-Pro-Leu-Gly-Ile-Ala-Gly-(Pro-Hyp-Gly)₄-OH (Sequence ID: 7) was synthesized by a solid-phase synthesis with an automatic peptide synthesis machine. That is, with the use of 0.1 mmol of a particulate resin [HMP glycine, manufactured by Applied Biosystems (US)] which comprised a styrene-divinylbenzene copolymer [molar ratio of styrene relative to divinylbenzene: 99/1] containing 4-(N^{α}-9-(fluorenylmethoxycarbonyl)-glycine)-oxymethyl-phenoxy-methyl group in a proportion of 0.65 mmol/g (resin), the carboxyl terminal of one amino acid was sequentially linked (or bound) to the amino terminal of the other amino acid so as to give an object peptide. In this link reaction, 1 mmol of N^{α}-9-(fluorenylmethoxycarbonyl)-L-proline [Fmoc proline], 1 mmol of N^{α}-9-(fluorenylmethoxycarbonyl)-glycine [Fmoc glycine], 1 mmol of N^{α}-9-(fluorenylmethoxycarbonyl)-L-leucine [Fmoc leucine], 1 mmol of N^{α}-9-(fluorenylmethoxycarbonyl)-L-isoleucine [Fmoc isoleucine], and 1 mmol of N^{α}-9-(fluorenylmethoxycarbonyl)-L-alanine [Fmoc alanine] (each manufactured by Applied Biosystems (US)), and 1 mmol of N^{α}-9-(fluorenylmethoxycarbonyl)-O-t-butyl-L-hydroxyproline [Fmoc hydroxyproline] (manufactured by Bachem AG) were used as amino acids in each linking step.

The peptide resin obtained by the above-mentioned manner was treated with 10 mL of trifluoroacetic acid containing 5% water for 3 hours. The resulting solution was added to diethyl ether to form a precipitate, and the precipitate was further washed with diethyl ether several times to deprotect the peptide and to eliminate the peptide from the resin. The resulting crude product was purified by a PD10 column (manufactured by Amarsham Bioscience K.K.) to give a peptide. The purified peptide obtained in the foregoing manner was subjected to a column chromatography [“AKTA explorer10XT” manufactured by Amarsham Bioscience K.K., column: “Nova-Pak C18”, manufactured by Millipore Corporation, 3.9 mmφ×150 mm, mobile phase: a mixed solvent of acetonitrile and water containing 0.05 vol. % of trifluoroacetic acid (concentration of acetonitrile was linearly increased from 5 to 50 vol. % for 30 minutes), flow rate: 1.0 mL/min.], and a single peak was shown at a retention time of 15 minutes. The molecular weight of the purified peptide was determined as 2666.3 based on FAB method mass spectrum (theoretical value: 2664.9).

The polypeptide (1.2 mg (0.00045 mmol)) of H-(Pro-Hyp-Gly)₄-Pro-Leu-Gly-Ile-Ala-Gly-(Pro-Hyp-Gly)₄-OH was dissolved in 0.25 mL of 10 mM phosphate buffer solution (pH7.4). To the peptide solution were added 0.12 mg (0.0009 mmol) of 1-hydroxybenzotriazole, and 15.7 mg (0.082 mmol) of 1-ethyl-3-(3-dimethylaminopropyl)-carbodiimide hydrochloride, and the resulting mixture was further stirred for 2 days at 20° C. The obtained reaction solution was diluted 10-fold with water, and the diluted solution was dialyzed against water for 3 days for removing a reagent (such as a condensing agent) and an unreacted monomer to give a polypeptide (IId). The proportion of the peptide unit (4) relative to the peptide unit (5) [(4)/(5)] was 8/1 (=about 88.9/11.1) (molar ratio).

The resulting polypeptide (IId) was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was confirmed in the range from 80,000 to 1,000,000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide (IId) was measured, and positive Cotton effect was observed at a wavelength of 224 nm and negative Cotton effect at a wavelength of 197 nm. The results confirmed that the polypeptide formed a triple helical structure.

### Example 1 (Bioapplicable Nonwoven Fabric)

The polypeptide (Ih) obtained in the Production Example 7 was diluted to have a concentration of about 20 mg/mL, and a polyglycol acid-series nonwoven fabric (Neoveil manufactured by Gunze Limited) having a size of 3 cm×3 cm was infiltrated into 0.5 mL of the diluted solution. Thereafter, the infiltrated fabric was freeze-dried to obtain a biomaterial in the form of a nonwoven fabric.

### Test Example 2

Liver of a Japanese white rabbit was exposed, and the ⅓ lower part of the right lobe of the liver was removed. After stanching by applying a pressure to the removed (transection) surface with a gauze, the nonwoven biomaterial obtained in the Example 1 was tightly put on the removed part, and the periphery of the biomaterial was fixed with a suture. The rabbit was killed at 3 months after operation, and the liver tissue including the removed part was extirpated. The liver tissue was visually observed and histologically investigated. The implanted biomaterial was completely absorbed with decomposition at 3 months after operation, and a visually and histologically normal liver tissue was regenerated. Further, there is no indication of scars, inflammatory reaction, and the like.

### Example 2 (Wound Dressing)

The polypeptide (Ih) obtained in the Production Example 7 was diluted to about 20 mg/mL, and 2.5 mL of the diluted solution was mixed with 2.5 mL of 1% by weight of aqueous solution of sodium alginate (manufactured by Kimica corporation, 99 mPa·s). The mixture was cast onto a fluorine resin tray having an inner size of 3 cm×3 cm, and the cast material was freeze-dried to obtain a sponge-like wound dressing.

### Test Example 3

A full thickness defective injury (8 mm in diameter) was made in each auricle of Japanese white rabbits, and the wound dressing obtained in the Example 2 was tightly put on the injury. On the wound dressing, was put gauze having a size of 3 cm×3 cm, and the gauze was fixed with an adhesive plaster. The rabbit was kept to feed for 10 days, thereafter the injury was observed. In the case of applying the wound dressings of the Example 2, all of 8 rabbits were closed and epithelialisation of the injuries was completed. On the contrary, in the case of not applying the wound dressings of the Example 2 but only covering the injuries with gauze, 3 rabbits out of 8 rabbits were not completely closed and epithelialisation of the injuries was also insufficient.

### Example 3 (Implant)

The aqueous solution of the polypeptide (IIc) obtained in the Production Example 10 was freeze-dried to make powdery. The powdery polypeptide (IIc) (100 mg) was mixed with 10 mL of 1% by weight of a normal saline solution of sodium alginate (manufactured by Kimica corporation, 99 mPa·s) to obtain a liquid implant.

### Test Example 4

After dorsal hair of a 6-week-old female Wistar rat was removed with a hair dipper and a dehairing cream, the liquid implant obtained in the Example 3 was injected to the several spots (0.3 mL/spot) in the dorsal derma. Thereafter, the implanted parts were visually observed over 8 weeks. Further, 8 weeks after the operation, the rat was killed, and the tissue of the implanted part was extirpated for histological investigation. In the visual observation, the swelling of the implanted part was gradually reduced over time, and almost disappeared at 8 weeks after the operation. The histological investigation of tissue revealed that the implants were almost completely absorbed with decomposition, and there is no indication such as traces of implant, scar tissues, or inflammatory reaction.

### Example 4 (Antiadhesive Material)

The polypeptide (IIb) solution (2.5 mL) obtained in the Production Example 9 was mixed with 1% by weight aqueous solution of sodium alginate (manufactured by Kimica corporation, 99 mPa·s). The mixture was cast onto a fluorine resin tray having an inner size of 3 cm×3 cm, and the cast material was air-dried at a room temperature to obtain a sheet-like antiadhesive material.

### Test Example 5

One side of the appendix of a 6-week-old male Wistar rat was abraded with dry gauze 6 times to make an abraded wound on the chronic membrane. The antiadhesive material obtained in the Example 4 was tightly put on the abraded wound. Two weeks after the operation, the rat underwent another abdominal operation, and the degree of coalescence (adhesion) was evaluated by the number of adhesions generated. In the case of using the sheet-like antiadhesive material obtained in the Example 4, the average number of adhesion sites was 1.3. On the contrary, the average number of adhesion sites was 3.5 in the case of not using the antiadhesive material.

### Example 5 (Adhesive Agent)

The aqueous solution of the polypeptide (IIa) obtained in the Production Example 8 was freeze-dried to give a powdery matter. The obtained powder (99 parts by weight) was well-mixed with N-succinimidyl 3-maleimidepropionato (manufactured by Aldrich) (1 part by weight), and a powdery adhesive agent was obtained.

### Test Example 6

The peritoneum of a 6-week-old male Wistar rat was exposed, and an incision wound about 1 cm in length was made in the central of the abdomen. The powdery adhesive agent obtained in the Example 5 was applied to the incision wound, and the incision wound was closed with tweezers and kept being closed for 5 minutes. Thereafter, the peritoneum cut into a size of 4 cm in length and 1 cm in width with centering the incision wound was removed. Immediately after removing, the tensile breaking strength of the removed membrane was measured with a tensile tester (manufactured by Instron Corp.). In the case of using the powdery adhesive agent obtained in Example 5, the tensile breaking strength of the peritoneums was 2 to 10 MPa. On the contrary, in the case of not using anything, the tensile breaking strength of all the peritoneums was not more than 1 MPa.

### Example 6 (Skin Lotion)

The aqueous solution of the polypeptide (Ih) obtained in the Production Example 7 was freeze-dried to give a powdery polypeptide. To 10 mL purified water were added 0.1 g of the powdery polypeptide, 0.1 g of polyethylene glycol (PEG 1500), 0.5 g of propylene glycol, and 0.5 g of glycerin, and the mixture was sufficiently stirred to obtain a solution. To the solution, was further added 1 mL of ethanol solution (containing 1% by weight of a paraben), and the resulting mixture was sufficiently stirred. The resulting mixture was diluted with purified water to 20 mL in total to prepare a skin lotion.

### Example 7 (Milky Lotion)

To 70 g of purified water were added 0.5 g of a powdery polypeptide which was the same with that used in the Example 6, 5.0 g of propylene glycol, 3.0 g of polyethylene glycol and 1.0 g of triethanolamine. The mixture was heated to have a liquid temperature of 70° C. to prepare a aqueous phase.

The mixture of 2.0 g of stearic acid, 1.0 g of cetyl alcohol, 2.0 g of a vaseline, 5.0 g of a squalan and 2.0 g of glycerol tri-2-ethylhexanate was heated to 75° C. for dissolving, and to the mixture were further added 2.0 g of sorbitan monooleate and 0.1 g of a paraben. The temperature of the obtained mixture was adjusted to 70° C. to prepare an oil phase.

To the aqueous phase, was added the oil phase, and the resulting mixture was stirred and mixed. Thereafter, the mixture containing emulsified particles was further homogenized with a homogenizer, and the homogenized emulsion was deaerated and filtrated. After cooling the resultant filtrate, a milky lotion was obtained.

### Example 8 (Moisturizing Essence)

To 14 g of purified water were added 0.4 g of a powdery polypeptide which was the same with that used in the Example 6, 1.6 g of sorbitol, 1.0 g of propylene glycol and 1.4 g of polyethylene glycol (PEG1500) to prepare an aqueous solution.

Besides, to 1.4 g of ethanol were added 0.2 g of polyoxyethylene oleyl alcohol ether, 40 mg of olive oil, and 20 mg of a paraben to prepare an ethanol solution. The aqueous solution was added to the ethanol solution and the both solutions were mixed to prepare a moisturizing essence.

### Example 9 (Hand Cream)

To 66.2 g of purified water were dissolved 0.5 g of a powdery polypeptide which was the same with that used in the Example 6, 15.0 g of glycerin, 3.0 g of propylene glycol and 0.2 g of potassium hydroxide. The mixture was heated to have a liquid temperature of 70° C. to prepare an aqueous phase.

A mixture of 3.0 g of stearic acid, 3.0 g of monoglyceride stearate, 3.0 g of a vaseline, 6.0 g of liquid paraffin and 0.1 g of a paraben was heated, and the temperature of the mixture was adjusted to 70° C. to prepare an oil phase.

The oil phase was added to the aqueous phase, and the resulting mixture was stirred and mixed. Thereafter, the stirred mixture containing emulsified particles was further homogenized with a homogenizer, and the homogenized emulsion was deaerated and filtrated. After cooling the resultant filtrate, a hand cream was obtained.

### Example 10 (Energy Drink)

By freeze-drying a polypeptide (Ih) obtained in the Production Example 7, a powdery polypeptide was obtained. To 100 mL of purified water were dissolved 0.02 g of the obtained powdery polypeptide, 0.1 g of vitamin C, 0.01 g of calcium lactate, 10 g of glucose, and 1 g of citric acid to give an energy drink.

### Example 11 (Dietary Supplement)

By freeze-drying a polypeptide (Ih) obtained in the Production Example 7, a powdery polypeptide was obtained. The obtained powdery polypeptide (0.05 g) was mixed with vitamin C (0.1 g), calcium lactate (0.1 g), and glucose fatty acid ester (0.1 g). The mixture was pressed to pelletize to give a dietary supplement.

### Test Example 7

Wistar type 6-week-old female rats were operated to remove ovaries, and used as an experimentally osteoporosis rat. Moreover, other Wistar type 6-week-old female rats were operated in the same manner with ovaries-removed rats except that ovaries-removing operation was not conducted after exposing ovaries, and used as a control (sham control). Three months after the operation, the experimentally osteoporosis rats were daily administered the energy drink (1 mL/day) obtained in the Example 10, or the dietary supplement (0.1 g/day) obtained in the Example 11. Three months after starting administration, a femur (thigh bone) of the rats was removed, and the bone density of a cortical bone in distal 1/10 from head of the femur was determined by the pQCT measurement.

The bone density of the sham control rat was 0.27 g/cm³, whereas the bone densities of the rats administered an energy drink and a dietary supplement both of which did not contain the polypeptide (Ih) obtained in the Production Example 7 were significantly decreased to 0.23 g/cm³ and 0.21 g/cm³, respectively. On the contrary, the bone densities of the rats administered the energy drink obtained in the Example 10 and the dietary supplement obtained in the Example 11 were significantly increased to 0.25 g/cm³ and 0.26 g/cm³, respectively.

### Example 12 (Tablet)

By freeze-drying a polypeptide (Ih) obtained in the Production Example 7, a powdery polypeptide was obtained. The obtained powdery polypeptide (0.05 g) was uniformly mixed with vitamin C (0.1 g), calcium lactate (0.1 g), glucose fatty acid ester (0.1 g). The mixture was pressed to pelletize to give a tablet.

### Test Example 8

Wistar type 6-week-old female rats were operated to remove ovaries, and used as an experimentally osteoporosis rat. Moreover, other Wistar type 6-week-old female rats were operated in the same manner with ovaries-removed rats except that ovaries-removing operation was not conducted after exposing ovaries, and used as a control (sham control). Three months after the operation, the experimentally osteoporosis rat was daily administered the tablet (amount of ⅓) obtained in the Example 12. Three months after starting administration, a femur (thigh bone) of the rat was removed, and the bone density in distal 1/10 from head of the femur was determined by the pQCT measurement. The bone density of the sham control rat was 0.27 g/cm³, whereas the bone density of a rat administered no tablet significantly decreased to 0.23 g/cm³. On the contrary, the bone density of the rat administered the tablet obtained in the Example 12 was significantly increased to 0.25 g/cm³.

### Example 13 (Injectable Preparation)

A polypeptide (Ih) obtained in the Production Example 7 was diluted to 20 mg/mL to give a polypeptide solution, and to 5 mL of the polypeptide solution was dissolved 0.1 mg of a basic fibroblast growth factor. The resultant solution was dropped into 50 mL of methylene chloride containing 0.05% of Span 80, and the mixture was stirred with a polytron homogenizer for 1 minute to be emulsified. The emulsified solution was freeze-dried to obtain a fine particulate injectable preparation (sustained release product for injection) containing the basic fibroblast growth factor.

### Example 14 (Paint)

By freeze-drying a polypeptide (Ih) obtained in the Production Example 7, a powdery polypeptide (powdery polypeptide (Ih)) was obtained. To a solution in which 10 g of an acrylic resin was dissolved in 30 mL of butyl acetate, was added 1 g of the powdery polypeptide, and the mixture was stirred to obtain a transparent paint. The obtained paint was excellent in adhesiveness to a substrate such as wood or timber.

### Example 15 (Coating Agent)

To a solution in which 10 g of a polyether-based polyurethane resin was dissolved in a mixture solution of 15 mL of methyl ethyl ketone and 15 mL of dimethylformamide, was added 1 g of a powdery polypeptide (Ih). The resultant mixture was stirred to obtain a coating agent. The obtained coating agent is high in adhesion to a substrate such as a leather or an artificial leather, and the coated material was also excellent in flexibility.

### Example 16 (Starch Agent)

To 20 mL of an aqueous solution containing a nonionic surfactant (polyoxyethylene alkyl phenyl ether) in a proportion of 0.3% by weight, was added 0.5 g of the powdery polypeptide (Ih), and the mixture was stirred to give a dispersion liquid containing the powdery polypeptide (Ih) (collagen). The dispersion liquid (20 g) and an aqueous solution (80 g) containing a hydroxypropylated starch (viscosity of 4% by weight solution at 20° C.: 10 mPa·s) in a proportion of 10% by weight were mixed and stirred to obtain a starch agent. The starch agent was well-adhered to a fiber product such as clothes, and a texture thereof was excellent.

### Example 17 (Adhesive Agent)

The mixture of 100 mL of water and 50 g of a powdery polypeptide (Ih) was subjected to an autoclave treatment (121° C., 10 minutes heating), and a semiliquid adhesive agent was obtained. The adhesive agent showed good adhesive strength to a substrate such as a fiber product.

What is claimed is:

1. A bioapplicable material containing a polypeptide, wherein the polypeptide comprises a synthetic polypeptide having at least an amino acid sequence represented by the formula: Pro-Y-Gly,
wherein Y represents Pro or Hyp,
and forming a collagen-like structure.

2. A bioapplicable material according to claim 1, wherein the polypeptide is at least one polypeptide selected from the group consisting of the following polypeptides (I) and (II):
(I) a polypeptide comprising peptide units represented by the following formulae (1) to (3):
[—(OC—(CH₂)ₘ—CO)ₚ-(Pro-Y-Gly)ₙ-]ₐ (1)
[—(OC—(CH₂)ₘ—CO)_{q}-(Z)ᵣ-]_{b} (2)
[—HN—R—NH—]_{c} (3)
wherein “m” denotes an integer of 1 to 18, “p” and “q” are the same or different, each representing 0 or 1, Y represents Pro or Hyp, and “n” denotes an integer of 1 to 20; Z represents a peptide chain comprising 1 to 10 amino acid residue(s), “r” denotes an integer of 1 to 20, and R represents a straight or branched alkylene group; the proportion (molar ratio) of “a” relative to “b” is 100/0 to 30/70,
when p=1 and q=0, c=a,
when p=0 and q=1, c=b,
when p=1 and q=1, c=a+b, and
when p=0 and q=0, c=0; and
(II) a polypeptide comprising a peptide unit having an amino acid sequence represented by the following formula (4) and a peptide unit having an amino acid sequence represented by the following formula (5):
-Pro-Y-Gly- (4)
wherein Y has the same meaning as defined above;
-Pro-V-Gly-W-Ala-Gly- (5)
wherein V represents Gln, Asn, Leu, Ile, Val or Ala, and W represents Ile or Leu.

3. A bioapplicable material according to claim 2, wherein
in the polypeptide (I), “m” denotes an integer of 2 to 12, “n” denotes an integer of 2 to 15, Z represents an amino acid or peptide chain comprising 1 to 10 amino acid residue(s) selected from the group consisting of Gly, Sar, Ser, Glu, Asp, Lys, H is, Ala, Val, Leu, Arg, Pro, Tyr and Ile, “r” denotes an integer of 1 to 10, and R represents a C₂₋₁₂alkylene group; and
the polypeptide (II) contains the peptide unit having an amino acid sequence represented by the formula (4) and the peptide unit having an amino acid sequence represented by the formula (5) in a molar ratio [(4)/(5)] of 99/1 to 30/70.

4. A bioapplicable material according to claim 2, wherein the polypeptide (I) comprises at least one member selected from the group consisting of the following polypeptides (i) to (iii):
(i) a polypeptide containing a unit represented by (Pro-Pro-Gly)ₙ,
(ii) a polypeptide containing a unit represented by (Pro-Hyp-Gly)ₙ, and
(iii) a polypeptide containing a unit represented by (Pro-Pro-Gly)ₙ₁ and a unit represented by (Pro-Hyp-Gly)ₙ₂,
wherein, in the polypeptides (i) to (iii), each of “n”, “n1” and “n2” represents a repeating number of each unit, “n1” plus “n2” is “n”; and
in the polypeptide (II), (iv) Y represents Hyp, V represents one residue selected from the group consisting of Gln, Asn, Leu, Ile, Val and Ala, W represents Ile or Leu, or (v) Y represents Pro, V represents one residue selected from the group consisting of Gln, Asn, Leu, Ile, Val and Ala, and W represents Ile or Leu.

5. A bioapplicable material according to claim 1, which shows positive Cotton effect at a wavelength in range of 220 to 230 nm and negative Cotton effect at a wavelength in range of 195 to 205 nm in a circular dichroism spectrum, and wherein at least part of the polypeptide is capable of forming a triple helical structure.

6. A bioapplicable material according to claim 1, which shows a peak of the molecular weight in the range from 5×10³ to 500×10⁴ in the molecular weight distribution.

7. A bioapplicable material according to claim 1, wherein the polypeptide is degradable with a collagenase.

8. A bioapplicable material according to claim 1, which is at least one member selected from the group consisting of a biomaterial or biocompatible material, a cosmetic preparation, a food composition, and a pharmaceutical preparation composition.

9. A bioapplicable material according to claim 1, which is a coating material, an implant material, a hemostatic material, an antiadhesive material, an adhesive material, a liniment or paint, a tube member, or a membrane material.

10. A bioapplicable material according to claim 1, which comprises a base, and a polypeptide recited in claim 1 applied on at least a surface of the base.

11. A bioapplicable material according to claim 10, wherein the base has biodegradability and biosorbability.

12. A bioapplicable material according to claim 1, which is a food or an animal feeding stuff.

13. A bioapplicable material according to claim 1, which is a sustained release pharmaceutical preparation.

14. A bioapplicable material according to claim 1, which is in the form of a powder, a solid, a semisolid, or a liquid.

15. A film-forming material which contains a polypeptide and is applied to a base, wherein the polypeptide comprises a synthetic polypeptide having at least an amino acid sequence represented by the formula: Pro-Y-Gly
wherein Y represents Pro or Hyp,
and forming a collagen-like structure.

16. A film-forming material according to claim 15, wherein the polypeptide is at least one polypeptide selected from the group consisting of the following polypeptides (I) and (II):
(I) a polypeptide comprising a peptide unit represented by the following formulae (1) to (3):
[—(OC—(CH₂)ₘ—CO)ₚ-(Pro-Y-Gly)ₙ-]ₐ (1)
[—(OC—(CH₂)ₘ—CO)_{q}-(Z)ᵣ-]_{b} (2)
[—HN—R—NH—]_{c} (3)
wherein “m” denotes an integer of 1 to 18, “p” and “q” are the same or different, each representing 0 or 1, Y represents Pro or Hyp, and “n” denotes an integer of 1 to 20; Z represents a peptide chain comprising 1 to 10 amino acid residue(s), “r” denotes an integer of 1 to 20, and R represents a straight or branched alkylene group; the proportion (molar ratio) of “a” relative to “b” is 100/0 to 30/70,
when p=1 and q=0, c=a,
when p=0 and q=1, c=b,
when p=1 and q=1, c=a+b, and
when p=0 and q=0, c=0; and
(II) a polypeptide containing a peptide unit having an amino acid sequence represented by the following formula (4) and a peptide unit having an amino acid sequence represented by the following formula (5):
-Pro-Y-Gly- (4)
wherein Y has the same meaning as defined above;
-Pro-V-Gly-W-Ala-Gly- (5)
wherein V represents Gln, Asn, Leu, Ile, Val or Ala, and W represents Ile or Leu.

17. A film-forming material according to claim 15, wherein the polypeptide shows a peak of the molecular weight in the range from 5×10³ to 500×10⁴ in a molecular weight distribution thereof.

18. A film-forming material according to claim 15, which is a coating agent or an adhesive.
